(19) United States
(12) Patent Application Publication
(10) Pub. No.: US 2022/0375617 A1
(43) Pub. Date: Nov. 24, 2022

(54) COMPUTERIZED DECISION SUPPORT TOOL FOR PREVENTING FALLS IN POST-ACUTE CARE PATIENTS

(71) Applicant: CERNER INNOVATION, INC., North Kansas City, MO (US)

(72) Inventors: Leonardo Ji, Overland Park, KS (US); Andrew Roberts, Overland Park, KS (US); Joseph O'Malley, Mission, KS (US)

(21) Appl. No.: 17/738,199

(22) Filed: May 6, 2022

Related U.S. Application Data
(60) Provisional application No. 63/186,101, filed on May 8, 2021.

(51) Int. Cl. G16H 50/30; G16H 50/20; G16H 10/60

(52) U.S. Cl. CPC G16H 50/30; G16H 50/20; G16H 10/60

(57) ABSTRACT

Systems, methods, and media are provided for predicting fall risk for a post-acute care patient. Patient data for the post-acute care patient is received. Features from the patient data are extracted. The features include one or more polypharmacy features. Other features may include lab or vital features. Based on the features extracted from the patient data, a prediction of the post-acute care patient suffering a fall within the future is generated using one or more machine learning models. The one or more machine learning models may be trained using one or more of binary features, continuous features, categorical features, free-text features, or a combination thereof. An action is initiated based on the prediction of the post-acute care patient suffering a fall. The action is associated with reducing the risk of the patient fall, such adjusting the lighting in the patient's room, for example.

### CROSS-REFERENCE TO RELATED APPLICATIONS

This application claims the benefit of U.S. Provisional App. No. 63/186,101, filed May 8, 2021, and titled “COMPUTERIZED DECISION SUPPORT TOOL FOR PREVENTING FALLS IN POST-ACUTE CARE PATIENTS.” The aforementioned application is assigned or under obligation of assignment to the same entity as this application, and is incorporated in its entirety in this application by reference.

### BACKGROUND

According to the CDC, falls among adults age 65 and older are very costly. Each year, in the US, an estimated $50 billion is spent on non-fatal fall injuries with that number expected to grow to $55 Billion by 2020. As of 2015, an estimated $754 million is spent on medical expenses from fatal falls ($28.9 billion is paid by Medicare, $8.7 billion is paid by Medicaid, and $12.0 billion is paid by private or out-of-pocket payers). For patients in an Inpatient or Skilled Nursing Facility, the estimated average added cost of fall resulting in an injury is $21,424, and the cost is not reimbursed by Centers for Medicare and Medicaid Services (CMS). Encompass has an average of 2,931 falls that resulted in an injury per year with an estimated cost of $62.8 million dollars.

Conventional technologies for predicting patient falls, including those that implement the Morse Fall Risk Scale, were developed for use in acute care settings. As such, they fail to provide accurate and appropriate decision support for caretakers in a post-acute care setting, such as an inpatient rehab facility (IRF). Particularly, these technologies were developed specifically for an acute care population, which tends to include different ages, pre-existing conditions, and mobility than patients in post-acute care settings. Additionally, conventional technologies are not suitable for use with post-acute care patients because the venue of care, such as services provided, length of stay, and physical space, differ between acute care and post-acute care settings. For example, in post-acute care facilities, the length of stay tends to be longer and there the space is geared towards rehabilitation and patients performing various activities, rather than being in a patient bed. As a result, there is a significant number of false positives in predicting falls for post-acute care patients using conventional technologies, which can lead to alert fatigue and inefficient use of resources. In using a technology implementing the Morse Fall Risk, for example, the area under the curve for an ROC graph is 0.5572 for post-acute care patients, which is only slightly better than tossing a coin. Additionally, conventional technologies do not utilize robust data to improve accuracy of the prediction or utilize data that is available earlier in a patient's stay, which increases the risk of missing a fall as 15.5% of all fall patients had a fall in the first 36 hours of their stay. Therefore, it is beneficial to predict early using available data, and deliver prediction early with a low latency pipeline. Accurately predicting patients at risk of having a fall event would enables efficient allocation of resources and initiating interventions for high-risk individuals—leading to both improved outcomes for patient health and safety as well as cost savings.

### BRIEF DESCRIPTION OF THE DRAWINGS

The present invention is described in detail below with reference to the attached drawing figures, wherein:

FIGS. 1A and 1B depict aspects of an illustrative operating environment suitable for practicing an embodiment of the disclosure; and

FIGS. 2-25 depict various other aspects of embodiments of this disclosure.

### DETAILED DESCRIPTION OF THE INVENTION

The subject matter of the present invention is described with specificity herein to meet statutory requirements. However, the description itself is not intended to limit the scope of this patent. Rather, the inventors have contemplated that the claimed subject matter might also be embodied in other ways, to include different blocks or combinations of blocks similar to the ones described in this document, in conjunction with other present or future technologies. Moreover, although the terms “step” and/or “block” may be used herein to connote different elements of methods employed, the terms should not be interpreted as implying any particular order among or between various blocks herein disclosed unless and except when the order of individual blocks is explicitly described.

In addition, words such as “a” and “an,” unless otherwise indicated to the contrary, include the plural as well as the singular. Thus, for example, the constraint of “a feature” is satisfied where one or more features are present. Furthermore, the term “or” includes the conjunctive, the disjunctive, and both (a or b thus includes either a or b, as well as a and b).

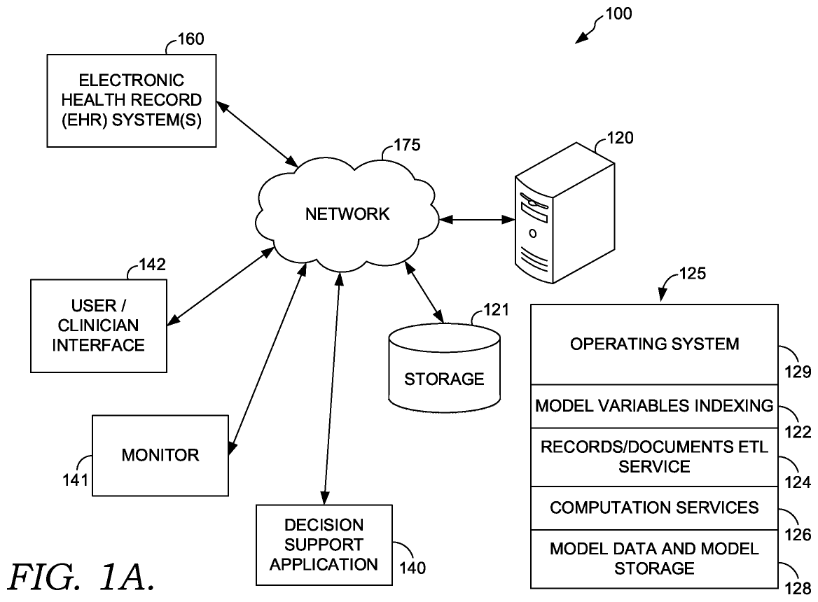

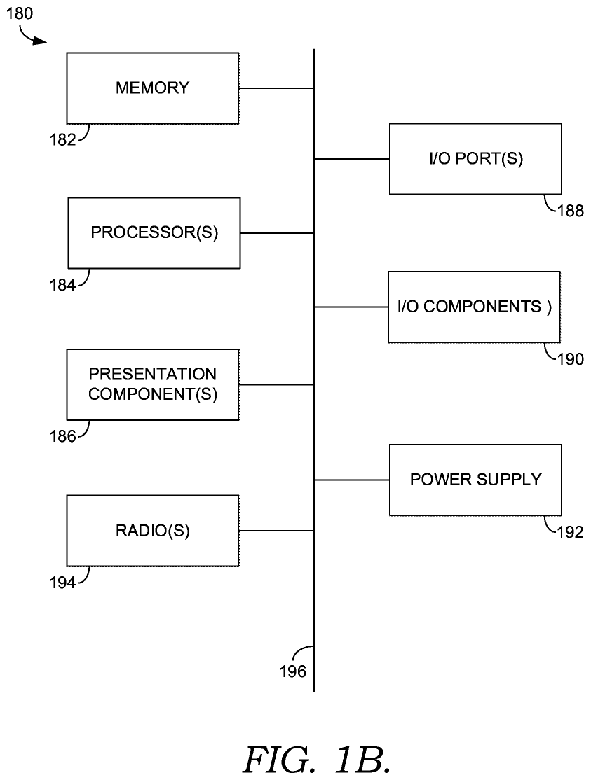

As one skilled in the art will appreciate, embodiments of the invention may be embodied as, among other things: a method, system, or set of instructions embodied on one or more computer-readable media. Accordingly, the embodiments may take the form of a hardware embodiment, a software embodiment, or an embodiment combining software and hardware. In one embodiment, the invention takes the form of a computer-program product that includes computer-usable instructions embodied on one or more computer-readable media, as discussed further with respect to FIGS. 1A-1B.

Accordingly, at a high level, this disclosure describes, among other things, methods and systems for predicting whether a patient in a post-acute care setting will have a fall within a future time period. In exemplary embodiments, the fall prediction is generated using one or more machine learning models with clinical and non-clinical patient data. Based on the fall prediction, one or more actions may be initiated. In some embodiments, the methods and systems may be implemented as a decision support computer application or tool for managing the patient's care while in the post-acute care setting. For instance, the fall prediction may trigger one or more actions to reduce the likelihood of a fall, including limiting the patient to less risky activities or rooms that do not require as much walking or steps, scheduling additional personnel to monitor the patient, or modifying medications to reduce the fall risk.

Embodiments of the fall risk prediction model may use features extracted from patient demographics, social determinants of health (SDOH) (e.g., presence of safe housing, education, job opportunities, polluted air and water, language skills, smoke-free living environment), inpatient medications (which may be medications administered), hospital services, billing diagnoses, functional assessments, cognitive assessments, labs, and/or vitals. The fall risk prediction model may be developed to predict a fall, such as a first fall, early in a post-acute care patient's encounter to ensure timely intervention and prevention. Additionally, the model may output only clinically meaningful features.

In some embodiments, the hospital service is combined with the billing diagnosis into a single feature for several common conditions, which may improve the model's performance (as indicated by AUC, for example). In some embodiments, natural language processing (NLP) techniques may be used to extract feature values from free text fields in the EMR, such as extracting a condition from a ‘Reason For Visit’ field. In one embodiment, NLP technique FastText is used to create word embeddings, UMAP is used for dimensionality reduction, and HDBSCAN is used to cluster. Performing NLP on a free text field, such as Reason For Visit may enable data that is accessible earlier to be used as a proxy for a primary diagnosis.

Some embodiments use polypharmacy features engineered for the fall risk prediction model. The polypharmacy features may count and change in counts of unique medications administered over various lookback periods (e.g., the last two days, the last three days, the last five days, the last week). Additionally, features relating to drug interactions for multiple medications administered to a patient may be extracted from the patient data and input into the fall risk prediction model. In one embodiment, XGBoost SHAP interaction values (sum of absolute values) are used to find pairs of drugs with highest interactions to test as combinations in feature selection. Additionally or alternatively, the Drug Burden Index (DBI) may be used to identify relevant medication features based on dosages. Additionally or alternatively, Multum drug interactions database may be accessed to identify relevant drug interactions.

The embodiments disclosed herein improve upon the conventional technologies by more accurately and more precisely predicting patient falls and initiating an action to reduce the patient's risk or likelihood of falling based on the more accurate and more precise prediction, which in turn results in more effective patient health services, thereby enhancing the quality of human life. Additionally, because conventional technologies only account for patients in acute care settings, the conventional technologies do not identify the appropriate data and do not analyze the appropriate data effectively for post-care patients. For example, in post-acute care facilities, embodiments of the present disclosure significantly reduce or eliminate the number of false positives in predicting falls for post-acute care patients, which results in the reduction or elimination of alert fatigue and more efficient use of resources.

The embodiments disclosed herein improve upon the area under the curve for an ROC graph used by the conventional technologies for predicting patient falls. For example, embodiments disclosed herein provide for using an XGBoost model for patient data associated with a post-acute care facility that improves the area under the curve for an ROC graph for predicting patient falls. Additionally, the embodiments disclosed herein provide for improved performance over Confusion matrices (e.g., based on optimal tpr-fpr) for logistic regression models and the XGBoost model used by the fall risk prediction model. Additionally, the particular methods of feature selection disclosed herein that are used for generating and employing the fall risk prediction model improve against biases that conventional technologies failed to consider, thereby further improving model performance of the fall risk prediction model.

Additionally, the embodiments disclosed herein improve upon the conventional technologies that do not utilize robust data by identifying received robust data and removing the data that does not have clinical relevance to implement analyses of relevant data from the large population of robust data. Further, improvements of the present technology that include faster processing of the robust data may occur as a result of standardizing patient data by using mappings that map client nomenclature and codes to standard nomenclature and codes, such as LONIC, SNOMED, ICD-10. The faster processing may also occur as a result of grouping the standardized codes and nomenclature into clinical ontology concepts that have the same or similar clinical meaning. Other improvements are discussed herein.

Referring now to the drawings generally and, more specifically, referring to FIG. 1A, an aspect of an operating environment 100 is provided for practicing an embodiment of this disclosure. The operating environment 100 is merely an example of one suitable operating environment and is not intended to suggest components that are not depicted may not be used. Neither should the operating environment 100 be interpreted as having a dependency or requirement relating to a single component or combination of components illustrated therein. Similarly, although some items are depicted in the singular form, plural items are contemplated as well (e.g., what is shown as one data store might really be multiple data-stores distributed across multiple locations); however, showing every variation of each item might obscure aspects of the invention. Thus, for readability, items are shown and referenced in the singular (while fully contemplating, where applicable, the plural).

As shown in FIG. 1A, example operating environment 100 provides an aspect of a computerized system for compiling and/or running an embodiment of a computer-decision support tool for example, predicting patient falls, include falls for post-acute care patients. Operating environment 100 includes one or more electronic health record (EHR) systems, such as hospital EHR system 160, communicatively coupled to network 175, which is communicatively coupled to computer system 120. In some embodiments, components of operating environment 100 that are shown as distinct components may be embodied as part of or within other components of operating environment 100. For example, EHR system 160 may comprise one or more EHR systems, such as hospital EHR systems, health information exchange EHR systems, ambulatory clinic EHR systems, and/or psychiatry/neurology EHR systems. Such EHR systems 160 may be implemented in computer system 120. Similarly, EHR system 160 may perform functions for two or more of the EHR systems (not shown).

Network 175 may comprise the Internet, and/or one or more public networks, private networks, other communications networks such as a cellular network, or similar network for facilitating communication among devices connected through the network 175. In some embodiments, network 175 may be determined based on factors such as the source and destination of the information communicated over network 175, the path between the source and destination, or the nature of the information. For example, intra-organization or internal communication may use a private network or virtual private network (VPN). Moreover, in some embodiments, items shown as being communicatively coupled to network 175 may be directly communicatively coupled to other items shown communicatively coupled to network 175.

In some embodiments, operating environment 100 may include a firewall (not shown) between a first component and network 175. In such embodiments, the firewall may reside on a second component located between the first component and network 175, such as on a server (not shown), or reside on another component within network 175, or may reside on or as part of the first component.

Embodiments of EHR system 160 include one or more data stores of health records, which may be stored on storage 121, and may further include one or more computers or servers that facilitate the storing and retrieval of health records. In some embodiments, EHR system 160 may be implemented as a cloud-based platform or may be distributed across multiple physical locations. EHR system 160 may further include record systems that store real-time or near real-time patient (or user) information, such as wearable, bedside, or in-home patient monitors, for example. Although FIG. 1A depicts an exemplary EHR system 160 that may be used for storing patient information, it is contemplated that an embodiment may also rely on decision support application 140 and/or monitor 141 for storing and retrieving patient record information, such as information acquired from monitor 141.

Example operating environment 100 further includes a provider user/clinician interface 142 communicatively coupled through network 175 to EHR system 160. Although operating environment 100 depicts an indirect communicative coupling between user/clinician interface 142 and EHR system 160 through network 175, it is contemplated that an embodiment of user/clinician interface 142 is communicatively coupled to EHR system 160 directly. An embodiment of user/clinician interface 142 takes the form of a graphical user interface operated by a software application or set of applications (e.g., decision support application 140) on a computing device. In an embodiment, the application includes the PowerChart® software manufactured by Cerner Corporation. In an embodiment, the application is a web-based application or applet. A healthcare provider application may facilitate accessing and receiving information from a user or healthcare provider about a specific patient or set of patients for which the likelihood of falling is predicted according to the embodiments presented herein. Embodiments of user/clinician interface 142 also facilitate accessing and receiving information from a user or healthcare provider about a specific patient or population of patients including patient history; healthcare resource data; physiological variables (e.g., vital signs) measurements, time series, and predictions (including plotting or displaying the determined fall risk prediction and/or issuing an alert) described herein; or other health-related information, and facilitates the display of results, recommendations, or orders, for example. In an embodiment, user/clinician interface 142 also facilitates receiving orders, such as orders for more resources, from a user based on the results of predictions. User/clinician interface 142 may also be used for providing diagnostic services or evaluation of the performance of various embodiments.

An embodiment of decision support application 140 comprises a software application or set of applications (which may include programs, routines, functions, or computer-performed services) residing on a client computing device, on one or more servers in the cloud, or distributed in the cloud and on a client computing device such as a personal computer, laptop, smartphone, tablet, mobile computing device, front-end terminals in communication with back-end computing systems or other computing device(s) such as computing system 120 described below. In an embodiment, decision support application 140 includes a web-based application or applet (or set of applications) usable to provide or manage user services provided by an embodiment of the invention. For example, in an embodiment, decision support application 140 facilitates processing, interpreting, accessing, storing, retrieving, and communicating information acquired from monitor 141, EHR system 160, or storage 121, including predictions and condition evaluations determined by embodiments of the invention as described herein. In an embodiment, decision support application 140 sends a recommendation or notification (such as an alarm or other indication) directly to user/clinician interface 142 through network 175. In an embodiment, application 140 sends a maintenance indication to user/clinician interface 142. In some embodiments, application 140 includes or is incorporated into a computerized decision support tool, as described herein. Further, some embodiments of application 140 utilize user/clinician interface 142. For instance, in one embodiment of application 140, an interface component, such as user/clinician interface 142, may be used to facilitate access by a user (including a clinician/caregiver or patient) to functions or information on monitor 141, such as operational settings or parameters, user identification, user data stored on monitor 141, and diagnostic services or firmware updates for monitor 141, for example.

In some embodiments, application 140 and/or interface 142 facilitates accessing and receiving information from a user or health care provider about a specific patient, a set of patients, or a population according to the embodiments presented herein. Such information may include historical data; health care resource data; variables measurements, time series, and predictions (including plotting or displaying the determined outcome and/or issuing an alert) described herein; or other health-related information. Application 140 and/or interface 142 also facilitates the display of results, recommendations, or orders, for example. In an embodiment, application 140 also facilitates receiving orders, scheduling time with care providers, or queries from a user, based on the results of the patient fall prediction, which may utilize user interface 142 in some embodiments.

Decision support application 140 may also be used for providing diagnostic services or evaluation of the performance of various embodiments. As shown in example environment 100, in one embodiment, decision support application 140, or the computer system on which it operates, is communicatively coupled to monitor 141 via network 175. In an embodiment, patient monitor 141 communicates directly (or via network 175) to computer system 120 and/or user/clinician interface 142. In an embodiment, monitor 141 (sometimes referred to herein as an patient-interface component) comprises one or more sensor components operable to acquire clinical or physiological information about a patient, such as various types of physiological measurements, physiological variables, or similar clinical information associated with a particular physical or mental state of the patient. Such clinical or physiological information may be acquired by monitor 141 periodically, continuously, as needed, or as they become available, and may be represented as one or more time series of measured variables. It is also contemplated that the clinical or physiological information about a patient or population of patients, such as the monitored variables, patient demographics, patient history, and/or clinical narratives regarding the patient, used according to the embodiment of the invention disclosed herein may be received from a patient's historical data in EHR system 160, or from human measurements, human observations, or automatically determined by sensors in proximity to the patient.

An embodiment of monitor 141 stores user-derived data locally or communicates data over network 175 to be stored remotely. In an embodiment, decision support application 140, or the computer system it is operating on, is wirelessly communicatively coupled to monitor 141. Application 140 may also be embodied as a software application or app operating on a user's mobile device, as described above. In an embodiment, application 140 and monitor 141 are functional components of the same device, such as a device comprising a sensor, application, and a user interface. In an embodiment, decision support application 140 is in communication with or resides on a computing system that is embodied as a base station, which may also include functionality for charging monitor 141 or downloading information from monitor 141.

Example operating environment 100 further includes computer system 120, which may take the form of a server, which is communicatively coupled through network 175 to EHR system 160, and storage 121. Computer system 120 comprises one or more processors operable to receive instructions and process them accordingly and may be embodied as a single computing device or multiple computing devices communicatively coupled to each other. In one embodiment, processing actions performed by computer system 120 are distributed among multiple locations such as one or more local clients and one or more remote servers and may be distributed across the other components of example operating environment 100. For example, a portion of computer system 120 may be embodied on monitor 141 or the computer system supporting application 140 for performing signal conditioning of a measured patient variable. In one embodiment, computer system 120 comprises one or more computing devices, such as a server, desktop computer, laptop, or tablet, cloud-computing device or distributed computing architecture, a portable computing device such as a laptop, tablet, ultra-mobile PC, or a mobile phone.

Embodiments of computer system 120 include computer software stack 125, which, in some embodiments, operates in the cloud as a distributed system on a virtualization layer within computer system 120, and includes operating system 129. Operating system 129 may be implemented as a platform in the cloud and is capable of hosting a number of services such as services 122, 124, 126, and 128, described further herein. Some embodiments of operating system 129 comprise a distributed adaptive agent operating system. Embodiments of services 122, 124, 126, and 128 run as a local or distributed stack in the cloud, on one or more personal computers or servers such as computer system 120, and/or a computing device running interface 142 and/or decision support application 140. In some embodiments, user/clinician interface 142 and/or decision support application 140 operate in conjunction with software stack 125.

In embodiments, model variables indexing service 122 provide services that facilitate retrieving frequent itemsets, extracting database records, and cleaning the values of variables in records. For example, service 122 may perform functions for synonymic discovery, indexing or mapping variables in records, or mapping disparate health systems' ontologies, such as determining that a particular medication frequency of a first record system is the same as another record system. In some embodiments, model variables indexing service 122 may invoke computation services 126. Predictive models service 124 is generally responsible for providing one or more models for predicting a fall for a post-acute care patient based on patient data for a plurality of dates to reduce the risk of fall as described further herein.

Computation services 126 perform statistical software operations. In an embodiment, computation services 126 and predictive models service 124 include computer software services or computer program routines. Computation services 126 also may include natural language processing services (not shown) such as Discern nCode™ developed by Cerner Corporation, or similar services. In an embodiment, computation services 126 include the services or routines that may be embodied as one or more software agents or computer software routines. Computation services 126 also may include services or routines for utilizing performing sequential modeling using one or more models, including decision trees and logistic models, for predicting a patient fall, such as the models described further herein.

In some embodiments, stack 125 includes file system or cloud-services 128. Some embodiments of file system/cloud-services 128 may comprise an Apache Hadoop and Hbase framework or similar frameworks operable for providing a distributed file system and which, in some embodiments, provide access to cloud-based services such as those provided by Cerner Healthe Intent®. Additionally, some embodiments of file system/cloud-services 128 or stack 125 may comprise one or more stream processing services (not shown). For example, such stream processing services may be embodied using IBM InfoSphere stream processing platform, Twitter Storm stream processing, Ptolemy or Kepler stream processing software, or similar complex event processing (CEP) platforms, frameworks, or services, which may include the use of multiple such stream processing services (in parallel, serially, or operating independently). Some embodiments of the invention also may be used in conjunction with Cerner Millennium®, Cerner CareAware® (including CareAware iBus®), Cerner CareCompass®, or similar products and services.

Example operating environment 100 also includes storage 121 (or data store 121), which, in some embodiments, includes patient data for a candidate or target patient (or information for multiple patients), including raw and processed patient data; variables associated with patient recommendations; recommendation knowledge base; recommendation rules; recommendations; recommendation update statistics; an operational data store, which stores events, frequent itemsets (such as “X often happens with Y,” for example), and itemsets index information; association rulebases; agent libraries, solvers and solver libraries, and other similar information including data and computer-usable instructions; patient-derived data; and healthcare provider information, for example.

In some embodiments, storage 121 includes training data for training one or more machine learning models, including one or more models for generating a prediction of a patient fall. In exemplary embodiments, an XGBoost model is utilized to predict a patient fall. In another embodiment, a logistic regression model is utilized to predict a patient fall. However, reference to these models is not intended to be limiting. For example, and without limitation, the one or more machine learning models may include other types of machine learning model, such as a machine learning model using linear regression, decision trees, support vector machines (SVM), Naïve Bayes, k-nearest neighbor (KNN), K means clustering, random forest, dimensionality reduction algorithms, neural networks (e.g., auto-encoders, convolutional, recurrent, perceptrons, Long/Short Term Memory (LSTM), Hopfield, Boltzmann, deep belief, deconvolutional, generative adversarial, liquid state machine, etc.), and/or other types of machine learning models.

Additionally, it is contemplated that the term “data” used herein includes any information that can be stored in a computer storage device or system, such as user-derived data, computer usable instructions, software applications, or other information. In some embodiments, storage 121 comprises data store(s) associated with EHR system 160. Further, although depicted as a single storage store, storage 121 may comprise one or more data stores, or may be in the cloud.

Turning briefly to FIG. 1B, there is shown one example embodiment of computing system 180 representative of a system architecture that is suitable for computer systems such as computer system 120. Computing system 180 includes a bus 196 that directly or indirectly couples the following devices: memory 182, one or more processors 184, one or more presentation components 186, input/output (I/O) ports 188, I/O components 190, radio 194, and an illustrative power supply 192. Bus 196 represents what may be one or more busses (such as an address bus, data bus, or combination thereof). Although the various blocks of FIG. 1B are shown with lines for the sake of clarity, in reality, delineating various components is not so clear, and metaphorically, the lines would more accurately be grey and fuzzy. For example, one may consider a presentation component, such as a display device, to be an I/O component. Also, processors have memory. As such, the diagram of FIG. 1B is merely illustrative of an exemplary computing system that can be used in connection with one or more embodiments of the present invention. Distinction is not made between such categories as “workstation,” “server,” “laptop,” “hand-held device,” etc., as all are contemplated within the scope of FIG. 1B and reference to “computing system.”

Computing system 180 typically includes a variety of computer-readable media. Computer-readable media can be any available media that can be accessed by computing system 180 and includes both volatile and nonvolatile media, and removable and non-removable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing system 180. Computer storage media does not comprise signals per se. Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

Memory 182 includes computer storage media in the form of volatile and/or nonvolatile memory. The memory may be removable, non-removable, or a combination thereof. Exemplary hardware devices include solid-state memory, hard drives, optical-disc drives, etc. Computing system 180 includes one or more processors that read data from various entities such as memory 182 or I/O components 190. Presentation component(s) 186 present data indications to a user or other device. Exemplary presentation components include a display device, speaker, printing component, vibrating component, etc.

In some embodiments, computing system 180 comprises radio(s) 194 that facilitates communication with a wireless-telecommunications network. Illustrative wireless telecommunications technologies include CDMA, GPRS, TDMA, GSM, and the like. Radio(s) 194 may additionally or alternatively facilitate other types of wireless communications including Wi-Fi, WiMAX, LTE, or other VoIP communications. As can be appreciated, in various embodiments, radio(s) 194 can be configured to support multiple technologies and/or multiple radios can be utilized to support multiple technologies.

I/O ports 188 allow computing system 180 to be logically coupled to other devices, including I/O components 190, some of which may be built in. Illustrative components include a microphone, joystick, game pad, satellite dish, scanner, printer, wireless device, etc. The I/O components 190 may provide a natural user interface (NUI) that processes air gestures, voice, or other physiological inputs generated by a user. In some instances, inputs may be transmitted to an appropriate network element for further processing. An NUI may implement any combination of speech recognition, stylus recognition, facial recognition, biometric recognition, gesture recognition both on screen and adjacent to the screen, air gestures, head and eye tracking, and touch recognition associated with a display of the computing system 180. The computing system 180 may be equipped with depth cameras, such as stereoscopic camera systems, infrared camera systems, RGB camera systems, touchscreen technology, and combinations of these, for gesture detection and recognition. Additionally, the computing system 180 may be equipped with accelerometers or gyroscopes that enable detection of motion.

The architecture depicted in FIG. 1B is provided as one example of any number of suitable computer architectures, such as computing architectures that support local, distributed, or cloud-based software platforms, and are suitable for supporting computer system 120.

Returning to FIG. 1A, in some embodiments, computer system 120 is a computing system made up of one or more computing devices. In some embodiments, computer system 120 includes one or more software agents and, in an embodiment, includes an adaptive multi-agent operating system, but it will be appreciated that computer system 120 may also take the form of an adaptive single agent system or a non-agent system. Computer system 120 may be a distributed computing system, a data processing system, a centralized computing system, a single computer such as a desktop or laptop computer or a networked computing system.

Aspects of this disclosure herein include predicting whether a post-acute care patient will fall within a future time interval. The fall prediction may be generated using one or more machine learning models from patient data. The patient data may be received from various sources, include the patient's electronic medical records. A patient's billing records are also utilized for retrieving patient data. In some embodiments, the patient data is received in JSON format as described further below. In some embodiments, raw patient data is received in a native nomenclature or code and translated into a standard nomenclature or code, such as LONIC, SNOMED, ICD-10.

From the patient data, which may be translated into a standard nomenclature or code, it may be predicted whether a particular patient in a post-acute care setting will have a patient fall. In exemplary aspects, prior to generating a prediction, the patient data may be transformed into engineered features, as explained further below. In exemplary aspects, the prediction as to whether a post-acute care patient will fall is determining a risk level that the patient will fall. In some aspects, the prediction is for a fall within a predetermined future time period, such as the next day, the next 36 hours, the next 3 days, the next week, or the next 15 days. The prediction may be generated using one or more machine learning models in which the patient data is input. One example machine learning model is a gradient boosting algorithm, such as XGBoost. Another example machine learning model is logistic regression. The prediction is output from the machine learning model(s). In some embodiments, an intervening action is initiated based on the prediction. The intervening action may be configured to reduce the risk of the patient fall, such adjusting the lighting in the patient's room, arranging for the patient to be located on a main level to avoid steps, scheduling additional monitoring, or allocating resources to the patient to prevent a fall.

Aspects of this disclosure include selecting the features from the patient data to input into the one or more machine learning models and training the machine learning model(s) to predict a patient fall risk. The training data may be standardized by using mappings that map client nomenclature and codes to standard nomenclature and codes, such as LONIC, SNOMED, ICD-10. The standard codes may be grouped into clinical ontology concepts that have the same or similar clinical meaning. In one embodiment, these concepts are reviewed by clinical experts. In one embodiment, training data is from a 14-month window (e.g., June 2019-July 2020). In one embodiment, the training data is received in JSON format from APACHE BEAM data pipeline.

In some embodiments, the data used in the machine learning model is encounter-based and certain criteria may be used to identify index episodes to be used for this training dataset. This criteria may include the patient being discharged being the appropriate prior window (e.g., Jun. 1, 2019-Jul. 30, 2020) and the patient has an “inpatient” encounter type. Additionally, the training data may be labeled as a fall or no fall. In some aspects, the labels may include unwitnessed, witnesses and intercepted, or witnessed but not intercepted. Of the “witnesses but not intercepted” falls, the training data may be labeled to include no injury, non-major injury, and major injury. In some embodiments, the training data included data for patients were intercepted and not intercepted and no injury, non-major injury, and major injury.

Table 1 provides a summary of the demographic and payer information for the patient data used to train the fall risk model.

TABLE 1

| | NO FALL | | FALL | | | |
|---|---|---|---|---|---|---|
| | n = 197711 | | n = 16307 | | TOTAL | |
| | (92.38%) | | (7.62%) | | n = 214018 | |
| AGE | 71.76 +/− 14.2 | | 69.37 +/− 14.28 | | 71.57 +/− 14.22 | |
| LENGTH OF STAY | 12.37 +/− 8.05 | | 16.71 +/− 10.02 | | 12.7 +/− 8.29 | |
| BIRTH GENDER | | | | | | |
| Male | 93984 | (47.54%) | 8794 | (53.93%) | 102778 | (48.02%) |
| Female | 103565 | (52.38%) | 7509 | (46.05%) | 111074 | (51.90%) |
| MATERNAL RACE/ETHNICITY | | | | | | |
| Caucasian | 154190 | (77.99%) | 12444 | (76.31%) | 166634 | (77.86%) |
| African American | 25214 | (12.75%) | 2328 | (14.28%) | 27542 | (12.87%) |
| Hispanic | 9266 | (4.69%) | 802 | (4.92%) | 10068 | (4.70%) |
| Other | 9053 | (4.58%) | 733 | (4.50%) | 9786 | (4.57%) |
| FINANCIAL CLASS | | | | | | |
| Medicare/Medicaid | 129220 | (65.36%) | 9507 | (5.30%) | 138727 | (64.82%) |
| Private Insurance | 12204 | (6.17%) | 1257 | (7.71%) | 13461 | (6.29%) |
| Other/Unknown | 56287 | (28.47%) | 5543 | (33.99%) | 61830 | (28.89) |

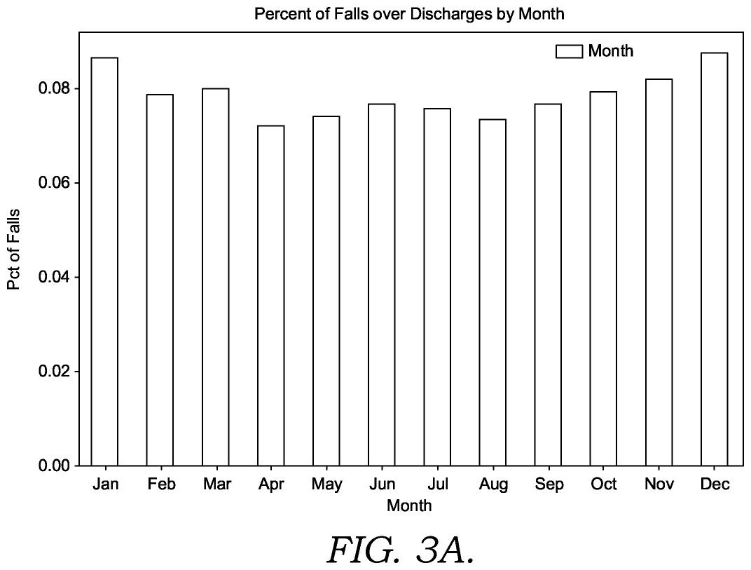

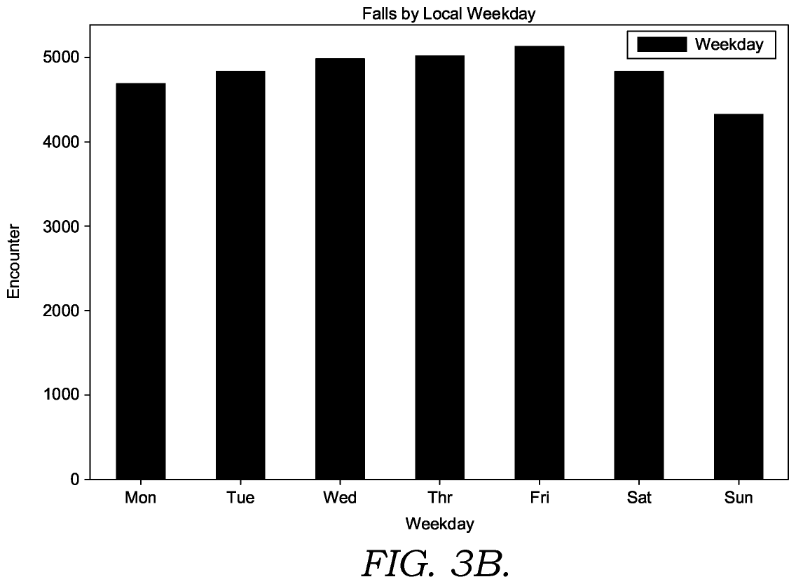

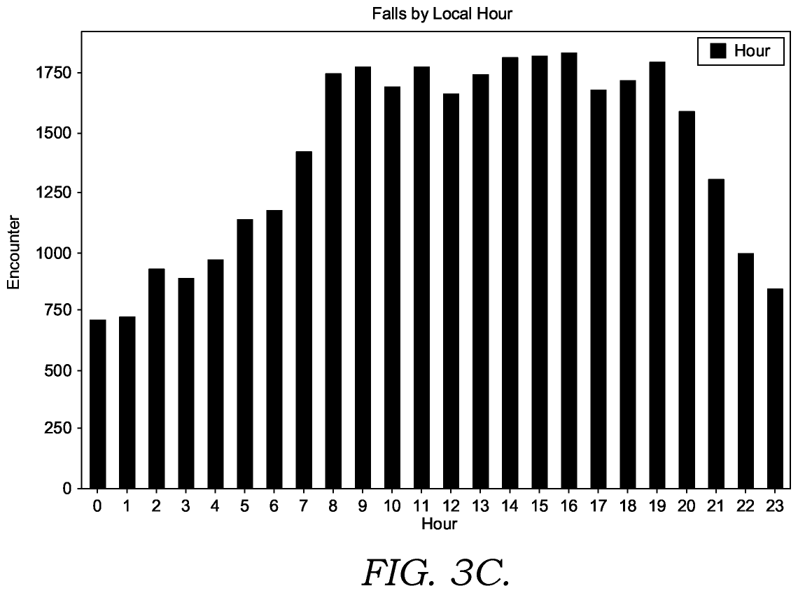

Table 1 shows that 7.6% of patient encounters had a fall with a majority being ‘unwitnessed.’ (Table 2, below shows the characteristics type of falls within an example training data set.) Additional analysis found that, 24.6% of the patients who fall have a second fall event. Off all of the fall events, 79% have no injury, 20% resulted in non-major injury, and 1% resulted in major injury. Aspects of this disclosure also considered time-based features, such distribution of falls by month (FIG. 3A), distribution of falls by the day of the week (FIG. 3B), and distribution of falls by the hour of the day (FIG. 3C).

Training of the one or more machine learning models, as well as utilization of the model(s) to predict a fall risk for a patient, may utilize a wide range of fields available in EMRs, including: diagnosis, medication orders, medication administrations, laboratory values, vitals, measures of functional abilities (e.g., Section GG), and several assessment fields typically included in an EMR, such as “Medical Service” provided (e.g., stroke program) and “Total Depression Screen score.” The format of data used in training and in deployment of the model may include binary features, continuous features, categorical features, and free-text features. In one embodiment, these fields in the EMR (shown in Table 2) include:

TABLE 2

| Variable Category | Description | Examples |
|---|---|---|
| Admission and | information describing the | Admission Source, Disposition of |
| discharge | discharge process under | Patient, Transferring to Rehabilitation, |
| information | various clinical settings | Length of Stay, Medical Service |
| | | provided, reason for visit |
| Assessment | Nursing Assessments typically | Affect, Appetite, Braden Scores, |
| | done at IRF hospitals | Sensory Braden, Functional Motor |
| | | Statuses, Pain Scores, History of Falls, |
| | | Bowel/Bladder Continence, Level of |
| | | Consciousness, Orientation, Total |
| | | depression score, IRF-PAI assessments, |
| | | Cognitive Functional Assessment . . . etc. |
| Behavioral/Cognitive | Any information describing | Smoking, Alcohol Use, Depression |
| | the patient's mental state of | Symptoms |
| | social behaviors exhibited | |
| Demographics | Demographic factors | Age, Gender, Race, Ethnicity |
| Diagnostic | The presence of a primary | ICD-10 codes, diagnostic categories |
| | diagnosis and/or co-morbidities | (working, primary, secondary, |
| | | discharge, principal) |
| Fall Outcome | The location/type of facility | Unwitnessed, Witnessed & Intercepted, |
| | where the patient is discharged | Caused minor injury |
| Indexes | Composite score compiled | Drug Burden Index |
| | Medication Administration | |
| Laboratory | Any laboratory codes, results | Albumin, ALT, AST, BUN, Chloride, |
| | or a cumulative number of | Creatinine Level, Glucose, Hematocrit, |
| | tests during index admission | Hemoglobin A1C, INR, Neutrophil |
| | | Count, O2 Sat, Platelet Count, |
| | | Potassium level, Serum Calcium, |
| | | Sodium Level, Troponin Level, |
| | | WBC . . . etc. |
| Medications | Presence of specific | Documented Medications, Inpatient |
| | medications, types of | Medications, Prescribed Medications, |
| | medications or the total | Medication Administrations |
| | number of medications the | |
| | patient is treated with | |
| Measures of | Section GG Functional | Lower Body Dressing Ability, Walk |
| Functional Mobility | Abilities (from CMS) | 150 Feet Ability |
| Vitals | Vital sign measurements | Blood Pressure, BMI, Carbon Dioxide, |
| | collected during the index | Heart Rate, Inhaled Oxygen |
| | admission | Concentration, Inhaled Oxygen Flow |
| | | Rate, Respiratory Rate, Pulse |
| | | Oximetry, Temperature |

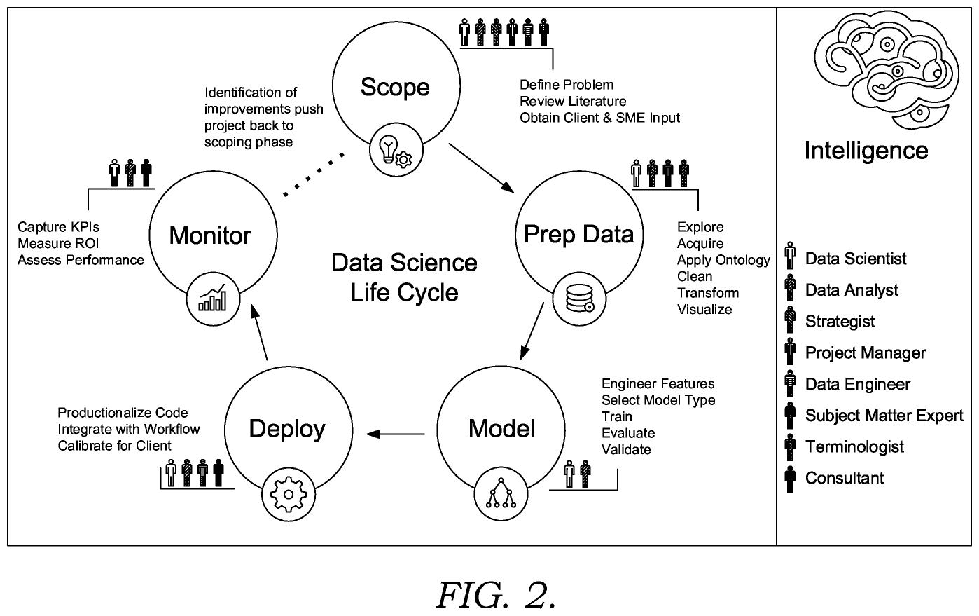

In some aspects, the model is developed in accordance with the Cross-Industry Standard Process for Data Mining (CRISP-DM) as shown in FIG. 2. The process may start with defining a positive target, data sources available, and deployment framework. From there, a clinical review of literature may be performed to find potential risk factors for further consideration. Iterative model versions may be produced based on the outcome of the clinical literature review, and with each model iteration, feedback would be provided to update the model, such as through removal of features that did not have clinical relevance, additional sources of data to look into that were being captured by clinicians, and additional or new instances of feature engineering.

In one embodiment, multiple types of models are developed. For example, a logistic regression model and an XGBoost model may both be developed. In some embodiments, the logistic regression model is used to target high-risk protocols based on why a patient was deemed high risk as the logistic regression model may provide interpretable coefficients. Otherwise, the XGBoost model may be utilized for increased accuracy.

In exemplary aspects, the model is trained based on data limited to an early stage of the patient encounter. Because over 50% of falls typically happen within the first 6 days, the model(s) may be trained to use data that is available early. As such, data beyond a pre-determined cutoff data may be removed from the training data. In one instance, this cutoff is selected from 0-3 days. Having relevant predictions early in the patient encounter allows time for preventive measures to be taken to before a fall happens.

In training and, in some aspects, deployment of the model, some patient data may be transformed. For continuous labs and vitals features, different values for unit of measurement may be converted into a single value of unit of measurement. Median imputation may be used for all continuous features with missing values, as it is less subject to outliers than mean. Additionally, some of the continuous features may be log transformed based on goal of keeping them in a normal distribution. For dealing with outliers in continuous result fields, winsorization may be applied based on clinically relevant ranges when present. When acceptable ranges were not present, Inner-Quartile Range (IQR) may be used. For example, any values beyond 1.5 IQR may be deemed outliers, and they may be appended to their respective edge of the range. Fields that contained such adjustments may include:
DIASTOLIC_BLOOD_PRESSURE between 40 and 100
CALCIUM_QUANTITATIVE_SERUM between 7.04 and 10.56
ALBUMIN in between 1.61 and 4.69
ALT_QUANTITATIVE_SERUM between 0 and 64.3
HEMATOCRIT_BLOOD between 13.07 to 52.23

For codified binary result features, some aspects use only those features with standardized qualifier value concept, such as the following:
APPETITE_STATUS
   GOOD_QUAL
   FAIR_QUAL
   POOR_QUAL
SENSORY_BRADEN
   SLIGHTLY_LIMITED
   VERY_LIMITED
   COMPLETELY_LIMITED
BLADDER_CONTINENCE
   STRESS_INCONTINENT
   NO_URINE_OUTPUT
   ALWAYS_CONTINENT
   OCCASIONALLY_INCONTINENT
   FREQUENTLY_INCONTINENT
   ALWAYS_INCONTINENT
UNDERSTANDING_OF_VERBAL_CONTENT
   USUALLY_UNDERSTANDS
   SOMETIMES_UNDERSTANDS
   RARELY_NEVER_UNDERSTANDS
LEVEL_OF_CONSCIOUSNESS
   ALERT
HALLUCINATIONS_PRESENT
   NONE
TWO_OR_MORE_FALLS_PAST_YEAR
   YES
   NO
ORIENTATION_ASSESSMENT
   INCONSISTENT
AFFECT
   CALM
   COOPERATIVE
   APPROPRIATE
ATTENTION_FUNCTIONAL_STATUS, COMPLEX_PROBLEM_SOLVING_FUNCTIONAL_STATUS, SIMPLE_PROBLEM_SOLVING_FUNCTIONAL_STATUS, MEMORY_FUNCTIONAL_STATUS, SAFETY_AWARENESS
   INDEPENDENT
   USUALLY_INDEPENDENT
   SOMETIMES_INDEPENDENT
   DEPENDENT
ABILITY_OBSTYPE
   DOES_NOT_OCCUR
   NOT_APPLICABLE
   PATIENT_REFUSED
   NOT_ATTEMPTED_DUE_TO_MEDICAL_CONDITION_OR_SAFETY_CONCERNS
   NOT_ATTEMPTED_DUE_TO_ENVIRONMENTAL_LIMITATIONS
   DEPENDENT
   SUBSTANTIAL_MAXIMAL_ASSISTANCE
   PARTIAL_MODERATE_ASSISTANCE
   SUPERVISION_OR_SETUP
   INDEPENDENT

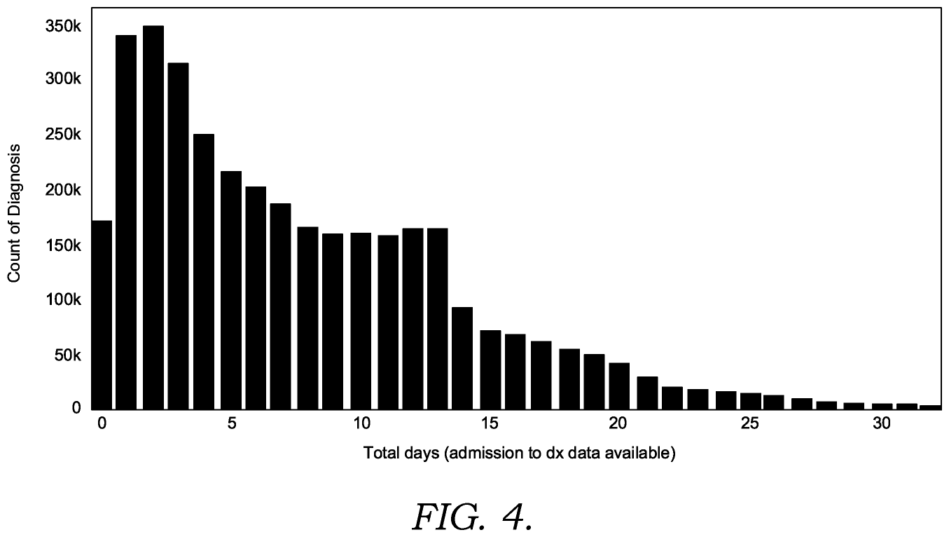

For binary condition features, some embodiment use type diagnosis, instead of problem. In general, billing diagnosis is considered more trustworthy than documented diagnosis or “Problem” in the patient EMR. As such, in some embodiments, diagnosis for model training and/or implementation may come from the billing record. FIG. 4 is a graphical depiction in the diagnosis count from a data set based on what day from the patient's admission the diagnosis became available. FIG. 4 shows that only 15.6 percent of all diagnosis are available in the first 48 hours after admission. As such, it is beneficial to use a proxy for diagnosis from the billing records that is available earlier in the patient's stay as described further below with respect to feature engineering.

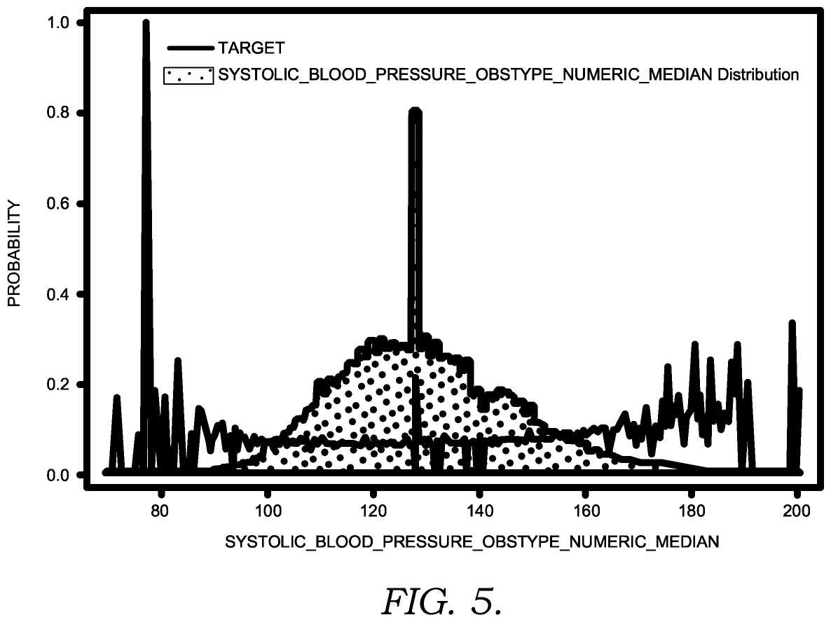

In an example embodiment reduced to practice using the patient data described above, conditional probability plots with distributions (counts) were determined from the data for continuous features of interest. These features included many labs and vitals (e.g. systolic blood pressure). Analyzing these distributions of binned continuous values provides confidence that there are not issues in the underlying data used in training to train an example fall prediction model (i.e. unit differences that need conversion). An example of the plot used is included in FIG. 5.

### Feature Engineering

In an example embodiment, the patient training data included ontology concepts and codified values pulled, that included 322 medication and medication groupings, 16 different medication counts, 211 diagnosis, 213 codified result fields, 32 assessment fields, and 558 different aggregations of labs/vitals, for a total of 1386 features.

In some embodiments, for continuous result features, feature engineering is performed using mean, median, maximum, minimum, change between first and last value, abnormal value (outside normal range), last value, last 48 hours mean value, last 48 hours change value. Some codified results, such as “appetite status”, may also be converted to numeric scores, and then the same logic above for the continuous features may be applied. Because historical medications from acute hospitals are typically automatically converted to inpatient medication orders in a post-acute care facility and some of the inpatient medications orders may not necessarily be given to the post-acute care patient, some embodiments utilize inpatient medication administrations data, rather than medication orders. Additionally, in exemplary embodiments, medication counts and/or aggregates (e.g., change in medications, cumulative counts) for lookback windows of different number of days (e.g., within the last 1 day, within the last 2 days, within the last 5 days, within the last week, within the last 2 weeks) may be created as features. For diagnoses, binary diagnosis features may be created such that if a patient is diagnosed with a condition, the diagnosis feature value may be a 1, otherwise the diagnosis feature value may be a 0. There may also be encounter-based features, such as length-of-stay (taken from admission datetime to current datetime in production). In one example embodiment, the training data included 618 binary features and 419 continuous features before feature selection.

Various embodiments of the disclosure include novel feature engineering techniques, including polypharmacy (patients with multiple administered medications), embedding and clustering of free text ‘Reason For Visit’, mobility devices data (wheelchair, scooters, no equipment), time of day, week, month, holidays, as well as others described herein.

Regarding polypharmacy feature engineering, some embodiments include engineering features from change in dosage and cumulative dosage of medications based on the Drug-Burden Index (DBI). Additionally, some embodiments of the disclosure include methods for finding specific medication interactions. The Multum interaction tables (‘mltm_int_drug_interactions’ and ‘mltm_interaction_description’) may be used to return descriptions for individual DNUM combinations, and features may be engineered for medication combinations with descriptions pertaining to falls (e.g., drowsiness, hallucinations, etc.). Additionally or alternatively, drug interactions may be found based purely on machine learning—using XGBoost with SHAP (SHapley Additive explanation) ‘TreeExplainer’ on a data sample (e.g., data for 100,000 encounters) for the full medication feature set. TreeExplainer, which is based on tree-based models, may isolate the gain in predictive value of features stand-alone as well as in combination. The medications with the largest difference (such as based on total variance), when looked at in combination compared to stand-alone, may then be included in feature selection.

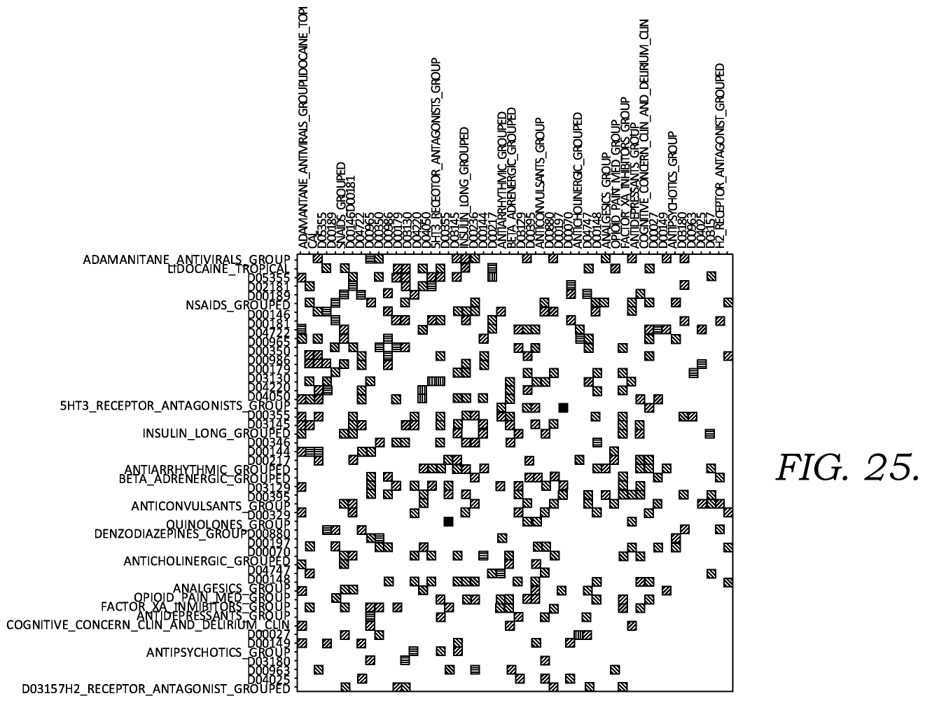

In some embodiments, only the total number of unique combinations of two medications are considered as processing all possible medication combinations for 322 unique medications over hundreds of thousands of encounters may not be computationally feasible. In some embodiments, these combinations may be determined from the following combination calculation:

$${C}_{( n , r )} = \frac{n !}{r ! ( n - r ) !}$$

where “C” represents total unique combinations, “n” represents unique medications (e.g., 322), and “r” represents the number of objects taken at a time (e.g., 2). FIG. 25 depicts a heat map that represents the interaction values and visualizes (on a sample) how medication combinations are identified. Lighter shades represent higher total interaction values.

In some embodiments, polypharmacy features may include a count of medications and/or whether or not there are four or more medications administered to a patient. In some aspects, the patient's age and the number of medications may be combined for a feature. For instance, patients having an age greater than 65 and a threshold number of medications (e.g., 2 medications) may be given a value of 1, otherwise a value of 0.

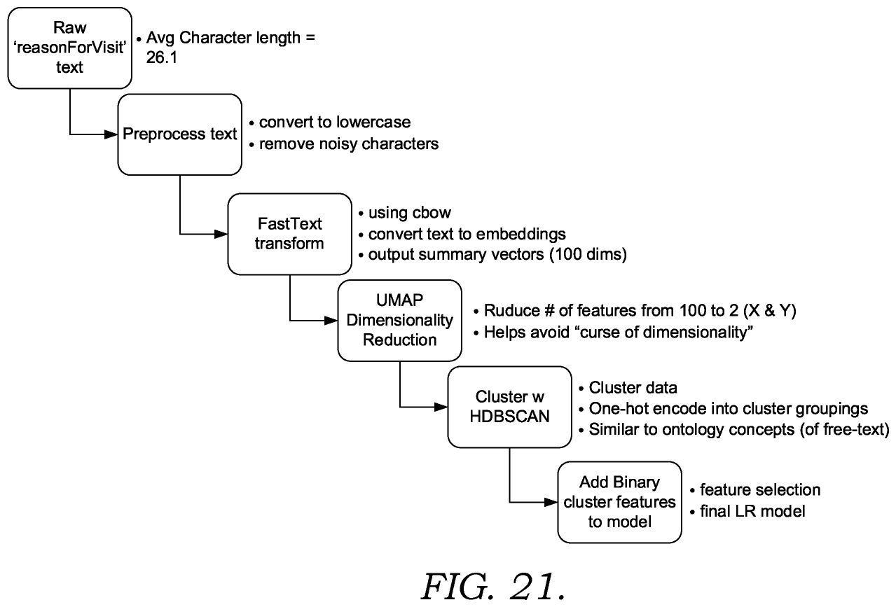

In exemplary aspects, the diagnosis may be determined from one or more free-text fields that clinicians and care managers fill out regularly in the normal course of basis. In one example, this information may come from a ‘Reason For Visit’ field attached to a patient's encounter. This ‘Reason For Visit’ field may hold relevant information, is available early for use in a model, and may have the most straight forward path to implementing in production. This field may contain free-text data on the patient's primary reason for being admitted to a post-acute care facility, such as a stroke, a hip fix, etc. To convert these free-text responses into a manageable feature set, embodiments may utilize one or more natural language process (NLP) techniques. In one example, an embedding approach called FastText is used to reduce the number of dimensions within the free text. Further, in some embodiments, clustering from the dimensions output by the embedding approach is used to make help make these dimensions interpretable for the final output of the model. This clustering approach may start reducing the embedded dimensions (e.g., FastText dimensions) to 3 dimensions by UMAP (or similar techniques) and clustered with HDBSCAN. The output of this process may be interpretable cluster assignments for feature selection and may be replicated as the fall prediction model sees new responses from the ‘Reason For Visit’ free text field. Because the ‘Reason For Visit’ field is available earlier in the patient's stay compared to a billing diagnosis, the ‘Reason For Visit’ may be used for condition feature as a proxy for diagnosis. FIG. 21 illustrates an example flow for feature engineering for a ‘Reason For Visit’ free text field. In some embodiments, other free-text fields, such as ‘History and Physical’ available in the patient data may be used by extracting features using NPL techniques such as FastText.

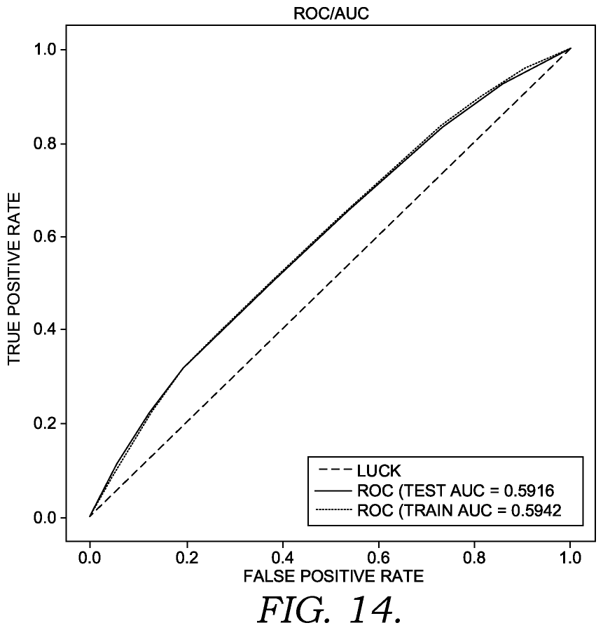

Some embodiments of this disclosure include creating and using a standalone model from the features engineered using only the ‘Reason for Visit’ field, which tends to be only 26.9 characters on average. FastText may be used to generate the embeddings. UMAP may be used to reduce dimensionality, and HDBSCAN may be used to create cluster assignments, which included 157 clusters in one embodiment. Based on these three processes, a model may be created with coefficients that could be loosely interpreted by looking at the free-text responses that fell into each (see example in Table 3, below). In one example embodiment, even with 40 percent of data not being assigned to a cluster, the Logistic Regression applied to the test set achieved an AUC of 0.592 (see FIG. 14).

TABLE 3
Free-Text Cluster Example

| Reason For Visit | unique_count |
|---|---|
| amputee lower ext | 630 |
| r bka | 450 |
| amputee lower ext | 428 |
| l bka | 396 |
| left bka | 280 |
| righ bka | 255 |
| l aka | 183 |
| r aka | 173 |
| left aka | 126 |
| right aka | 125 |
| rt bka | 55 |
| lt bka | 46 |
| amputee lower extremity | 8 |
| amputee program | 7 |

In some embodiments, an additional or alternative proxy for diagnosis may be a ‘Medical Service’ codified value. These medical services are typically available as the patient is admitted and closely align with the primary condition a patient is being seen for. This feature may be engineered using logic that accepts the codified value for medical service if it is present or diagnosis for the same condition if diagnosis is present. As such, medical service code may be combined with same ‘Billing’ diagnosis to create a condition features (patient with stroke program medical service code or STROKE diagnosis). Table 4 below shows example codified values for various medical services.

TABLE 4
Example Medical Services

| | Prevalence |
|---|---|
| Medical Service | (percent) |
| Neurological Condition | 20.07 |
| Stroke Program | 18.73 |
| Other Conditions | 11.54 |
| Orthopedic - Other | 8.34 |
| Orthopedic - HIP | 7.71 |
| Brain Injury - Non-Trauma | 7.69 |
| Cardiac Program | 4.51 |
| Mult TR No Brain/SCN | 4.28 |
| Orthopedic - Joint | 3.00 |
| Spinal Cord Non-Trauma | 2.94 |
| Amputee-Lower Extremity | 2.86 |
| Brain Injury - Trauma | 2.74 |
| Mult TR w/Brain/SCT | 1.32 |
| Pulmonary Program | 1.32 |
| Spinal Cord Injury -Trauma | 0.88 |
| None | 0.45 |
| Ortho -Osteoarthritis | 0.37 |
| Arthritis | 0.33 |
| Guillian-Barre | 0.27 |
| Pain Management | 0.26 |
| Rehabilitation | 0.12 |
| Internal Medicine | 0.10 |
| Burn Program | 0.07 |
| Amputee - Other | 0.07 |
| Skilled Nursing | 0.04 |
| Nephrology | 0.00 |

### Feature Selection

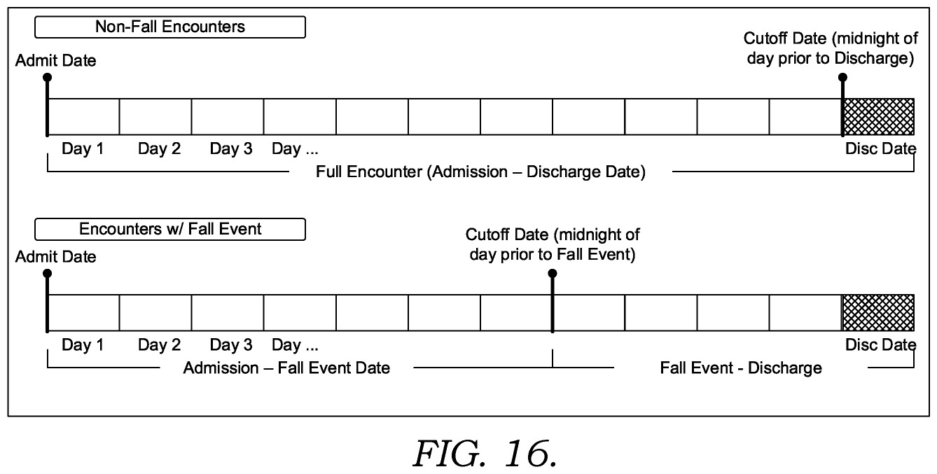

In exemplary embodiments, a sequential process is used to eliminate features from the full set of engineered features. In each step, less important or unstable features may be removed. In the first steps, the follow features may be removed:
Patient identifiable information including empi id, encounter id, patient names, aliases.
Data not available before target date (see FIG. 16).
Features not tried to specific IRF encounter in a valid IRF facility.
Conditions that are type problem.
Any binary features that are very sparse or populated in less than 0.1% in prevalence.
Any lab log transformed or not log transformed that is not in a uniform distribution.

In one embodiment reduced to practice, after these above filters, there were 992 features left to go through a model-based feature selection process. In some aspects, the feature selection process is based on XGBoost then GLMnet. Features with greater than 95 percent correlation (Pearson's Correlation, keeping the feature with less correlation to the rest of the data) may be dropped. Mean absolute correlations may be used to determine which feature to drop. When two features are correlated to each other, the feature that is more correlated to the rest of features is dropped.

Next, continuous and binary variables may be separated for each of the demographics, condition, medication, and result features. This step may be particular useful if using XGBoost because XGBoost is a tree-based learner and is biased toward features with high cardinality. Therefore, XGBoost may be performed on continuous and binary feature separately. In addition, randomly permuted features are added for each continuous feature. This step may be done before the actual XGBoost step. In some aspects, Bayesian optimization for XGBoost is used to tune parameters max depth, learning rate, min child weight, subsample, and gamma that gives higher AUC.

Following adding the addition of permuted features, two separate 10-fold XGBoost models may be applied on continuous and binary features for each of the demographics, condition, medication, and results features. This step may be for performance reasons and identify features from different types of data. Different parameter tunings and optimizations may be considered for XGBoost feature selection, including the choice between information gain and total gain as the selection criteria. The total gains are calculated and sorted on each feature. The permuted feature with the highest total gain may be identified as cutoff point to remove any features with less than the permuted feature total gain cutoff value. The remaining features may then be counted. This process may be done 10 times, and total counts of each features may be determined. Any features that are counted as being present in more than 90% of the models may be kept.

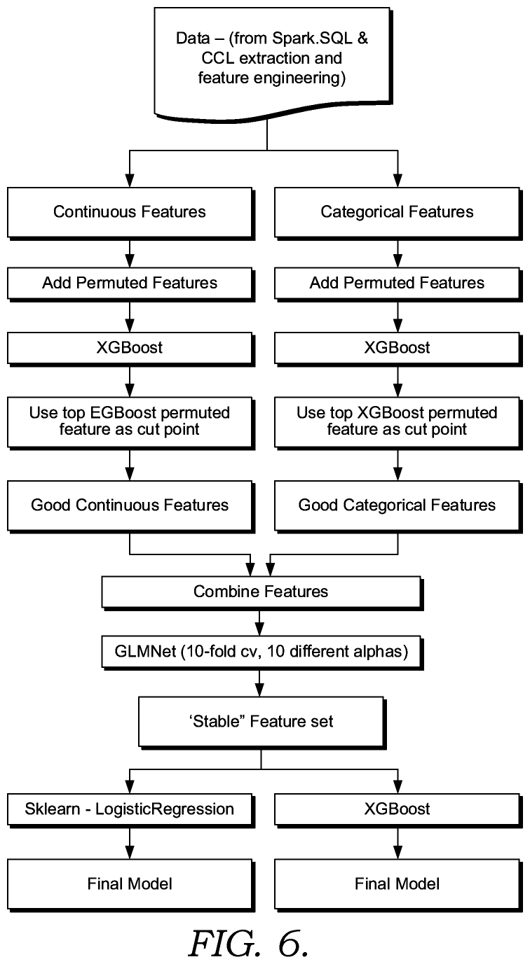

After the XGBoost step, the remaining binary features and continuous features may be combined in the next step using the GLMnet model. This is because the GLMnet is not a tree-based model and is thus not biased towards continuous or categorical features. After removing permuted features from the previous step, a 10-fold penalized logistic regression model (Adaptive-Logitnet) with 10 different alphas (elastic-net mixing parameter—between 0.01 and 0.99) may be applied. This model may keep “stable” features that show up in at least 90 percent of the runs (see FIG. 6). This GLMnet model applies adaptive penalties on both Lasso L1 and Ridge L2 regularizations and uses a 10-fold cross validation to determine the optimal regularization parameter lambda value. After feature eliminations, the final set of features may then be used to create the prediction model. There are total of 56 final features selected for the prediction model. The two final models are packaged as ‘tar.gz’ pickle files from the fitting of the final feature set with XGBoost and Sci-kit Learn Logistic Regression. The deployment pipeline disclosed below allows these two model to be switched in production.

### Feature Descriptions

Feature selection may be performed as described herein. In one embodiment, a total of 56 features are selected for use in the train fall risk prediction model. In exemplary aspects, when the model is run, not all of these features need be present in patient data to obtain a fall risk output. Each of the example features below may be can be classified into conditions, medications, patient history features, labs/vitals (results), assessment features, combination and composite features.

### Patient History Features

The following features based on patients' hospitalization history may be used with the fall risk prediction model:
Length of Stay, current
   length of stay, measured in days
   model may calculate this feature based on time delta between admission and target date
Gender (Male)
   Gender in “MALE” ontology concept
      “Falls Risk Prediction: 921b4db3-e797-4d46-8c82-aef56acfb7fc” ontology context
History of Falls, current
   Feature Name: “TWO_OR_MORE_FALLS_PAST_YEAR_CODIFIED_LAST”
   Observation Type in “TWO_OR_MORE_FALLS_PAST_YEAR” ontology concept
   “Falls Risk Prediction: 921b4db3-e797-4d46-8c82-aef56acfb7fc” ontology context
   Returns most recently recorded value
   Binary feature flagged if result >0

### Conditions Features

The example features in this section may be based directly on the diagnoses associated with the patient record. For diagnoses, Ontology Concepts may be used to map disparate codes to a common clinical interpretation (e.g. ICD-9, ICD-10). In exemplary aspects, the conditions considered are associated with the current encounter, rather than a past encounter.
Traumatic Brain Injury
   Diagnosis in “TRUMATIC_BRAIN_INJURY” ontology concept
      “Falls Risk Prediction: 921b4db3-e797-4d46-8c82-aef56acfb7fc” ontology context

### Combination Features

These example features in this section may be based directly on the diagnoses associated with the patient record. These features may consider whether the patient has one diagnoses from a group of diagnoses. In some aspects, to qualify as a feature, an encounter must meet at least one of the qualifying criteria:
Have a diagnosis associated with the current encounter. For diagnoses, Ontology Concepts were used to map disparate codes to a common clinical interpretation (e.g. ICD-9, ICD-10).
Have medical service associated with condition of interest for current encounter.

### Cerebrovascular Accident or Stroke Program Service

Feature name: “CEREBROVASCULAR_ACCIDENT_CLIN_or_hospitalService_Stroke_Program”
Diagnosis in “CEREBROVASCULAR_ACCIDENT” ontology concept
   “Falls Risk Prediction: 921b4db3-e797-4d46-8c82-aef56acfb7fc” ontology context
Assessment in “STROKE_PROGRAM_SERVICE” ontology concept
   “Falls Risk Prediction: 921b4db3-e797-4d46-8c82-aef56acfb7fc” ontology context

### Lower Extremity Amputation or Lower Extremity Rehab Service

Feature name: “LOWER_EXTREMITY_AMPUTATION_CLIN_or_hospitalService_Amputee_Lower_Extremity”
Diagnosis in “LOWER_EXTREMITY_AMPUTATION” ontology concept
   “Falls Risk Prediction: 921b4db3-e797-4d46-8c82-aef56acfb7fc” ontology context
Assessment in “AMPUTEE_LOWER_EXTREMITY_REHAB_SERVICE” ontology concept
   “Falls Risk Prediction: 921b4db3-e797-4d46-8c82-aef56acfb7fc” ontology context

### Cognitive Concern or Delirium or Brain Injury Non-Trauma Service

Feature name: “COGNITIVE_CONCERN_CLIN_AND_DELIRIUM_CLIN_or_hospitalService_Brain_Injury_Non_Trauma”
Diagnosis in “COGNITIVE_CONCERN” ontology concept
   “Falls Risk Prediction: 921b4db3-e797-4d46-8c82-aef56acfb7fc” ontology context
Diagnosis in “DELIRIUM” ontology concept
   “Falls Risk Prediction: 921b4db3-e797-4d46-8c82-aef56acfb7fc” ontology context
Assessment in “BRAIN_INJURY_NONTRAUMA_SERVICE” ontology concept
   “Falls Risk Prediction: 921b4db3-e797-4d46-8c82-aef56acfb7fc” ontology context

### Spinal Cord Non-Trauma Service or Spinal Cord Injury Trauma Service

Feature name: “hospitalService_Spinal_Cord_Non_Trauma_or_hospitalService_Spinal_Cord_Injury_Trauma”
Assessment in “SPINAL_CORD_NONTRAUMA_SERVICE” ontology concept
   “Falls Risk Prediction: 921b4db3-e797-4d46-8c82-aef56acfb7fc” ontology context
Assessment in “SPINAL_CORD_INJURY_TRAUMA_SERVICE” ontology concept
   “Falls Risk Prediction: 921b4db3-e797-4d46-8c82-aef56acfb7fc” ontology context

### Medications Features

The 28 example features in this section may be based directly on medications associated with the patient record. An additional 2 features may be engineered from medication counts. For medications, Ontology concepts may be used to map disparate codes to a common clinical interpretation (e.g. ICD-9, ICD-10, SNOMED). In exemplary embodiments, only medications that were administered during the patient stay qualifies as a feature for the fall risk prediction model.

### Furosemide, Administered

Medication in “D00070” ontology concept
   “Healthe Intent Medication: 13a95538-1fd0-4e23-8bec-48d851f04ff9” ontology context

### Gamma-Aminobutyric Acid Analogs (Group), Administered

Medication in “GAMMAAMINOBUTYRIC_ACID_ANALOGS_GROUP” ontology concept
   “Healthe Intent Medication: 13a95538-1fd0-4e23-8bec-48d851f04ff9” ontology context

### Laxatives (Group), Administered

Medication in “LAXATIVES_GROUP” ontology concept
   “Healthe Intent Medication: 13a95538-1fd0-4e23-8bec-48d851f04ff9” ontology context

### Adamantane Antivirals, Administered

Medication in “ADAMANTANE_ANTIVIRALS_GROUP” ontology concept
   “Healthe Intent Medication: 13a95538-1fd0-4e23-8bec-48d851f04ff9” ontology context
      Multivitamin with Minerals, Administered
Medication in “D03145” ontology concept
   “Healthe Intent Medication: 13a95538-1fd0-4e23-8bec-48d851f04ff9” ontology context

### Losartan, Administered

Medication in “D03821” ontology concept
   “Healthe Intent Medication: 13a95538-1fd0-4e23-8bec-48d851f04ff9” ontology context

### Thiamine, Administered

Medication in “D03130” ontology concept
   “Healthe Intent Medication: 13a95538-1fd0-4e23-8bec-48d851f04ff9” ontology context

### Levothyroxine, Administered

Medication in “D00278” ontology concept
   “Healthe Intent Medication: 13a95538-1fd0-4e23-8bec-48d851f04ff9” ontology context

### Albuterol Ipratropium, Administered

Medication in “ALBUTEROL_IPRATROPIUM” ontology concept
   “Falls Risk Prediction: 921b4db3-e797-4d46-8c82-aef56acfb7fc” ontology context

### Anti-Infectives, Administered

Medication in “ANTIINFECTIVE_MEDICATIONS” ontology concept
   “Falls Risk Prediction: 921b4db3-e797-4d46-8c82-aef56acfb7fc” ontology context

### Anticonvulsants (Group), Administered

Medication in “ANTICONVULSANTS_GROUP” ontology concept
   “Healthe Intent Medication: 13a95538-1fd0-4e23-8bec-48d851f04ff9” ontology context

### Anxiolytics, Sedatives, and Hypnotics (Group), Administered

Medication in “ANXIOLYTICS_SEDATIVES_AND_HYPNOTICS_GROUP” ontology concept
   “Healthe Intent Medication: 13a95538-1fd0-4e23-8bec-48d851f04ff9” ontology context

### Anticoagulants (Group), Administered

Medication in “ANTICOAGULANTS_GROUP” ontology concept
   “Healthe Intent Medication: 13a95538-1fd0-4e23-8bec-48d851f04ff9” ontology context

### Scopolamine, Administered

Medication in “D00986” ontology concept
   “Healthe Intent Medication: 13a95538-1fd0-4e23-8bec-48d851f04ff9” ontology context

### Analgesics (Group), Administered

Medication in “ANALGESICS_GROUP” ontology concept
   “Healthe Intent Medication: 13a95538-1fd0-4e23-8bec-48d851f04ff9” ontology context

### Cholecalciferol, Administered

Medication in “D03129” ontology concept
   “Healthe Intent Medication: 13a95538-1fd0-4e23-8bec-48d851f04ff9” ontology context

### Levothyroxine, Administered

Medication in “D00278” ontology concept
   “Healthe Intent Medication: 13a95538-1fd0-4e23-8bec-48d851f04ff9” ontology context

### Antihistamines (Group), Administered

Medication in “ANTIHISTAMINES_GROUP” ontology concept
   “Healthe Intent Medication: 13a95538-1fd0-4e23-8bec-48d851f04ff9” ontology context

### Fluticasone Nasal, Administered

Medication in “D04283” ontology concept
   “Healthe Intent Medication: 13a95538-1fd0-4e23-8bec-48d851f04ff9” ontology context

### Simethicone, Administered

Medication in “D01027” ontology concept
   “Healthe Intent Medication: 13a95538-1fd0-4e23-8bec-48d851f04ff9” ontology context

### Miscellaneous Analgesics (Group), Administered

Medication in “MISCELLANEOUS_ANALGESICS_GROUP” ontology concept
   “Healthe Intent Medication: 13a95538-1fd0-4e23-8bec-48d851f04ff9” ontology context

### Quinolones (Group), Administered

Medication in “QUINOLONES_GROUP” ontology concept
   “Healthe Intent Medication: 13a95538-1fd0-4e23-8bec-48d851f04ff9” ontology context

### Nicotine, Administered

Medication in “D00316” ontology concept
   “Healthe Intent Medication: 13a95538-1fd0-4e23-8bec-48d851f04ff9” ontology context

### Antiemetic/Antivertigo Agents, Administered

Medication in “ANTIEMETICANTIVERTIGO_AGENTS_GROUP” ontology concept
   “Healthe Intent Medication: 13a95538-1fd0-4e23-8bec-48d851f04ff9” ontology context

### Central Nervous System (Group), Administered

Medication in “CENTRAL_NERVOUS_SYSTEM_GROUP” ontology concept
   “Falls Risk Prediction: 921b4db3-e797-4d46-8c82-aef56acfb7fc” ontology context

### Long-Acting Insulin (Group), Administered

Medication in “INSUILIN_LONG_GROUP” ontology concept
   “Falls Risk Prediction: 921b4db3-e797-4d46-8c82-aef56acfb7fc” ontology context

### Antiarrhythmics (Group), Administered

Medication in “ANTIARRHYTHMIC_GROUP” ontology concept
   “Falls Risk Prediction: 921b4db3-e797-4d46-8c82-aef56acfb7fc” ontology context

### Opioids (Group), Administered

Medication in “OPIOID_PAIN_MED_GROUP” ontology concept
   “Falls Risk Prediction: 921b4db3-e797-4d46-8c82-aef56acfb7fc” ontology context

### Lidocaine Topical, Administered

Medication in “D00683” ontology concept
   “Healthe Intent Medication: 13a95538-1fd0-4e23-8bec-48d851f04ff9” ontology context
      Medication Count (Last day), Administered
Feature Name: “count_medications_chg_last_1_days”
Count of medications with administration in past 24 hours
   Medication Count (Last 2 days), Administered
Feature Name: “count_medications_chg_last_2_days”
Count of medications with administration in past 24 hours

### Laboratory/Vitals Features

In one example, five different laboratory and vitals values were used in building six features for the fall risk prediction model. These values may come from the “results” table from the EMR source in the longitudinal health record. Ontology concepts may be used with several different combinations of aggregates and lookback windows engineered and used in the feature selection process. In exemplary embodiments, Labs/Vitals are only used as features in the fall risk prediction model if they are associated with the current encounter for a patient.

### Albumin, Current (Maximum Value)

Feature: “ALBUMIN_NUMERIC_MAX”
“ALBUMIN” ontology concept
Returns Maximum recorded value associated with current encounter
“Falls Risk Prediction: 921b4db3-e797-4d46-8c82-aef56acfb7fc” ontology context
Values outside 1.61-4.69 range are winsorized (outliers appended to edges of range)

### Diastolic Blood Pressure, Last 48 Hours (Average Value)

Feature name: “DIASTOLIC_BLOOD_PRESSURE_NUMERIC_MEAN_LAST_48_HR”
“DIASTOLIC_BLOOD_PRESSURE” ontology concept
Returns mean of values captured in past 48 hours
“Falls Risk Prediction: 921b4db3-e797-4d46-8c82-aef56acfb7fc” ontology context
Values outside 40-100 range are winsorized (outliers appended to edges of range)

### Hematocrit—Blood, Last 48 Hours (Average Value)

Feature name: “HEMATOCRIT_OBSTYPE_NUMERIC_MEAN_LAST_48_HR”
“HEMATOCRIT_BLOOD” ontology concept
Returns mean of values captured in past 48 hours
“Falls Risk Prediction: 921b4db3-e797-4d46-8c82-aef56acfb7fc” ontology context
Values outside 13.07-52.23 range are winsorized (outliers appended to edges of range)

### Diastolic Blood Pressure, Current (Median Value)

Feature name: “DIASTOLIC_BLOOD_PRESSURE_NUMERIC_MEDIAN_CAPTURE”
“DIASTOLIC_BLOOD_PRESSURE” ontology concept
Returns median for all values captured during current encounter
“Falls Risk Prediction: 921b4db3-e797-4d46-8c82-aef56acfb7fc” ontology context
Values outside 40-100 range are winsorized (outliers appended to edges of range)
In some embodiments, this feature indicate whether current diastolic blood pressure is less than 55 mmHg and/or greater than 85 mmHg.

### Alanine Aminotransferase Test (SGPT) Quantitative Serum, Current (Last (Date) Value)

Feature name: “ALT_QUANTITATIVE_SERUM_NUMERIC_LAST_LOG”
“ALT_QUANTITATIVE_SERUM” ontology concept
May use Log transformation, Returns most recently recorded value
“Falls Risk Prediction: 921b4db3-e797-4d46-8c82-aef56acfb7fc” ontology context
Values outside 0-64.3 range are winsorized (outliers appended to edges of range)

### Calcium Quantitative Serum, Current (Last (Date) Value)

Feature name: “CALCIUM_QUANTITATIVE_SERUM_NUMERIC LAST”
“CALCIUM_QUANTITATIVE_SERUM” ontology concept
Returns most recently recorded value
“Falls Risk Prediction: 921b4db3-e797-4d46-8c82-aef56acfb7fc” ontology context
Values outside 7.04-10.56 range are winsorized (outliers appended to edges of range)

### Assessments Features

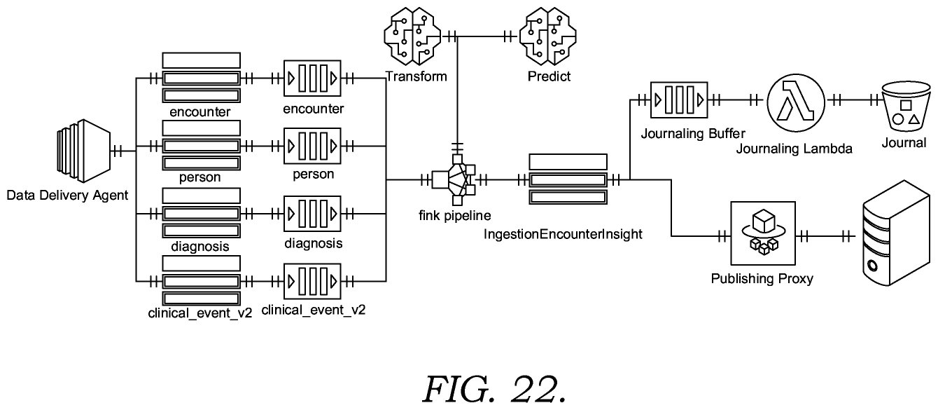

In some embodiments, there may be 15 different assessments be used to create 11 different assessments features for the fall risk prediction model. In exemplary aspects, assessments are used as features only if they are associated with the current encounter. FIG. 22

### Appetite Status

Feature Name: “APPETITE_STATUS_CODIFIED_LAST”
“APPETITE_STATUS_OBSTYPE” ontology concept
“Falls Risk Prediction: 921b4db3-e797-4d46-8c82-aef56acfb7fc” ontology context
Returns most recently recorded value
Qualifier concepts include: “GOOD”, “FAIR”, “POOR”

### Total Depression Screen Score

Feature Name: “TOTAL_DEPRESSION_SCREEN_SCORE_NUMERIC_LAST”
“TOTAL_DEPRESSION_SCREEN_SCORE” ontology concept
“Falls Risk Prediction: 921b4db3-e797-4d46-8c82-aef56acfb7fc” ontology context
Returns most recently recorded value
Binary feature flagged if result >0

### Sensory Braden

Feature Name: “SENSORY_BRADEN_CODIFIED_LAST”
“SENSORY_BRADEN” ontology concept
“Falls Risk Prediction: 921b4db3-e797-4d46-8c82-aef56acfb7fc” ontology context
Returns most recently recorded value
Qualifier concepts include: “SLIGHTLY_LIMITED”, “COMPLETELY_LIMITED”, “VERY_LIMITED”

### Braden Score

Feature Name: “BRADEN_SCORE_NUMERIC_MAX”
“BRADEN_SCORE” ontology concept
“Falls Risk Prediction: 921b4db3-e797-4d46-8c82-aef56acfb7fc” ontology context
Returns maximum value during current encounter
Binary feature flagged if result >0

### Affect

Feature Name: “AFFECT_CODIFIED_LAST”
“AFFECT” ontology concept
“Falls Risk Prediction: 921b4db3-e797-4d46-8c82-aef56acfb7fc” ontology context
Returns most recently recorded value
Qualifier concepts include: “CALM”, “COOPERATIVE”, “APPROPRIATE”

### Cognitive Functional Status (Composite Score)

Feature Name: “COGNITIVE_FUNCTIONAL_STATUS_SCORE_LAST”
“ATTENTION_FUNCTIONAL_STATUS”, “COMPLEX_PROBLEM_SOLVING_ABILITY”, “SIMPLE_PROBLEM_SOLVING_ABILITY”, “MEMORY_FUNCTIONAL_STATUS”, “SAFETY_AWARENESS” ontology concepts
“Falls Risk Prediction: 921b4db3-e797-4d46-8c82-aef56acfb7fc” ontology context
Returns codified sum for each concept's most recently recorded value
Qualifier concepts include: “INDEPENDENT”, “USUALLY_INDEPENDENT”, “SOMETIMES_INDEPENDENT”, “DEPENDENT”

### Understanding of Verbal Content

Feature Name: “UNDERSTANDING_OF_VERBAL_CONTENT_CODIFIED_LAST”
“UNDERSTANDING_OF_VERBAL_CONTENT” ontology concept
“Falls Risk Prediction: 921b4db3-e797-4d46-8c82-aef56acfb7fc” ontology context
Returns most recently recorded value
Qualifier concepts include: “USUALLY_UNDERSTANDS”, “SOMETIMES_UNDERSTANDS”, “RARELY_NEVER_UNDERSTANDS”

### Bowel Continence Status

Feature Name: “BOWEL_CONTINENCE_STATUS_CODIFIED_LAST”
“BOWEL_CONTINENCE_STATUS” ontology concept
“Falls Risk Prediction: 921b4db3-e797-4d46-8c82-aef56acfb7fc” ontology context
Returns most recently recorded value
Qualifier concepts include: “ALWAYS_CONTINENT”, “OCCASIONALLY_INCONTINENT”, “ALWAYS_INCONTINENT”

### Orientation Assessment

Feature Name: “ORIENTATION_ASSESSMENT_CODIFIED_LAST”
“ORIENTATION_ASSESSMENT” ontology concept
“Falls Risk Prediction: 921b4db3-e797-4d46-8c82-aef56acfb7fc” ontology context
Returns most recently recorded value
Qualifier concepts include: “INCONSISTENT”

### Bladder Continence

Feature Name: “BLADDER_CONTINENCE_CODIFIED_LAST”
“BLADDER_CONTINENCE” ontology concept
“Falls Risk Prediction: 921b4db3-e797-4d46-8c82-aef56acfb7fc” ontology context
Returns most recently recorded value
Qualifier concepts include: “ALWAYS_CONTINENT”, “STRESS_INCONTINENCE”, “NO_URINE_OUTPUT”

### Level of Consciousness or Hallucinations Present

Feature Name: “LEVEL_OF_CONSCIOUSNESS_CODIFIED_LAST_or_HALLUCINATIONS_PRESENT_CODIFIED_LAST”
“LEVEL_OF_CONSCIOUSNESS”, “HALLUCINATIONS_PRESENT” ontology concepts
“Falls Risk Prediction: 921b4db3-e797-4d46-8c82-aef56acfb7fc” ontology context
Returns most recently recorded value, binary flag if either codified concept is true
Qualifier concepts include: “NONE”, “ALERT”

### Functional Motor Features

In some embodiments, functional motor features are constructed using the weighted sum of functional abilities, which may be 23 unique fields, pertaining to Activities of Daily Living (ADLs). Each component may be rated on a 0 to 6 (Independent) scale. CMS changed requirements for of ADLs to require use of “Section GG” in October 2019. The new section GG may be used for functional motor features as well and it contains many similar ADLs that were available in FIM as well as several net new fields.

### GG Functional Abilities

‘SHOWER_BATHE_ABILITY’
   Imputed Value: this feature may have an inputed value as indicated below
‘PUT_ON_OFF_FOOTWEAR_ABILITY’
   Imputed Value: this feature may have an inputed value as indicated below
   Value multiplied 0.5
‘LOWER_BODY_DRESSING_ABILITY’
   Imputed Value: this feature may have an inputed value as indicated below
   Value multiplied 0.5
‘UPPER_BODY_DRESSING_ABILITY’
   Imputed Value: this feature may have an inputed value as indicated below
‘EATING_ABILITY’
   Imputed Value: this feature may have an inputed value as indicated below
‘ORAL_HYGIENE_ABILITY’
   Imputed Value: this feature may have an inputed value as indicated below
‘TOILETING_ABILITY’
   Imputed Value: this feature may have an inputed value as indicated below
‘CHAIR_BED_TRANSFER_ABILITY’
   Imputed Value: this feature may have an inputed value as indicated below
‘TOILET_TRANSFER_ABILITY’
   Imputed Value: this feature may have an inputed value as indicated below
Ability to Use Stairs, Last
   ‘TWELVE_STEPS_ABILITY’
   ‘ONE_STEP_ABILITY’ multiplied by 0.667
   ‘FOUR_STEPS_ABILITY’ multiplied by 0.334
   Imputed Value: this feature may have an inputed value as indicated below
Ability to Walk, Last
   ‘WALK_150_FEET_ABILITY’
   ‘WALK_50_FEET_2_TURNS_ABILITY’ multiplied by 0.667
   ‘WALK_10_FEET_ABILITY’ multiplied by 0.334
   Imputed Value: this feature may have an inputed value as indicated below
Ability to Wheel, Last
   ‘WHEEL_150_FEET_ABILITY’
   ‘WHEEL_50_FEET_2_TURNS_ABILITY’ multiplied by 0.667
   Imputed Value: this feature may have an inputed value as indicated below
‘LYING_TO_SITTING_ABILITY’
   Imputed Value: this feature may have an inputed value as indicated below
‘WALK_UNEVEN_10_FEET_ABILITY’
   Imputed Value: this feature may have an inputed value as indicated below
‘SITTING_TO_LYING_ABILITY’
   Imputed Value: this feature may have an inputed value as indicated below
‘SIT_TO_STAND_ABILITY’
   Imputed Value: this feature may have an inputed value as indicated below
‘ROLL_LEFT_AND_RIGHT_ABILITY’
   Imputed Value: this feature may have an inputed value as indicated below
‘PICKING_UP_OBJECT_ABILITY’
   Imputed Value: this feature may have an inputed value as indicated below
‘CAR_TRANSFER_ABILITY’
   Imputed Value: this feature may have an inputed value as indicated below

### Codified Values for Each Qualifier Concept:

6.0: ‘INDEPENDENT’
5.0: ‘SETUP_OR_CLEAN_UP_ASSISTANCE’
4.0: ‘SUPERVISION_OR_TOUCHING_ASSISTANCE’
3.0: ‘PARTIAL_MODERATE_ASSISTANCE’
2.0: ‘SUBSTANTIAL_MAXIMAL_ASSISTANCE’
1.0: ‘DEPENDENT’
0.0: ‘PATIENT_REFUSED’, ‘NOT_ATTEMPTED_DUE_TO_MEDICAL_CONDITION’
None: ‘NOT_APPLICABLE’, ‘NOT_ATTEMPTED_DUE_TO_ENVIRONMENTAL_CONSTRAINTS’, ‘DOES_NOT_OCCUR’

### Imputation: Median Imputation for Each Subcomponent Applied for Each ‘None’

### Model Calibration

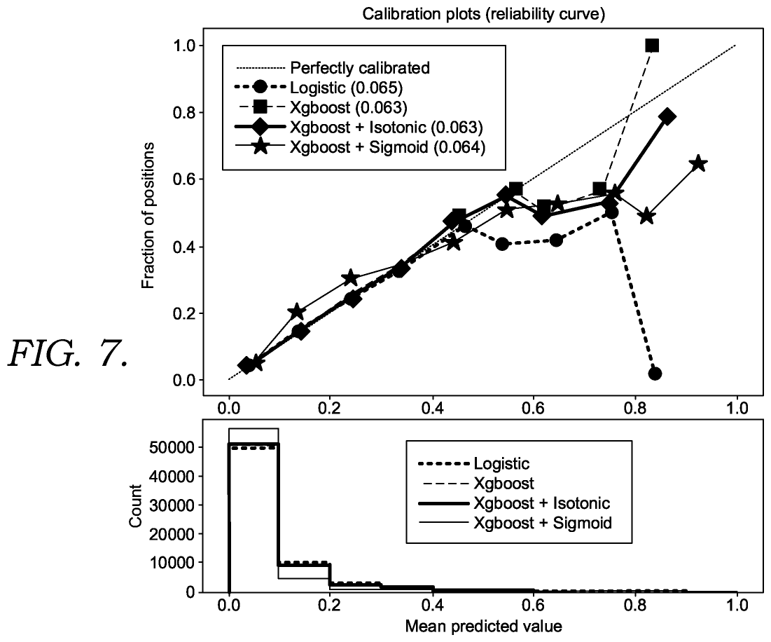

FIG. 7 shows the calibration curve for each of the two final models in one embodiment reduced to practice. The non-parametric approach of the Isotonic model and parametric approach Sigmoid model approaches are not necessary for Logistic Regression but are shown in FIG. 7 as comparisons. The calibration curve was created on the training data set and represents the predicted risk that the final Sci-Kit Learn Logistic Regression and XGBoost models produced, binned to increase ease of interpreting density of the predictions. The calibration curve may be used to set an appropriate and useful risk threshold when applying the fall prediction model in practice. Setting the risk threshold too high may lead to many missed falls, and setting it to low may lead to alert fatigue for end users. As such, understanding the differences at each of the client's facilities, distributions of high-risk patents, and volumes at each given threshold may be useful. In one example embodiment, the high risk threshold is set for greater than 0.2, a basic risk (low risk) threshold is set of 0.08-0.2, and a standard (no risk) threshold is set of less than 0.08. As such, if a patient has a fall risk prediction of 0.25, the patient may be designated as having a high fall risk and particular intervening and preventive actions may be taken for the individual. It is contemplated that other risk thresholds may be used.

### Model Validation and Performance

In some embodiments, the fall risk prediction model is validated through production of daily output in silent model that may be used to assess performance. The predictions (e.g., risk scores) from these reports on an individual patient basis may be reviewed manually to make sure the model workflow is behaving as expected. Further, there may be continuous automated monitoring of the model outputs to ensure that the model does not degree to an intolerable degree. For example, there may be safeguards in place to quit surfacing risk scores if the model performance does not meet a configurable threshold limit (e.g., if the AU-ROC is less than 0.65).

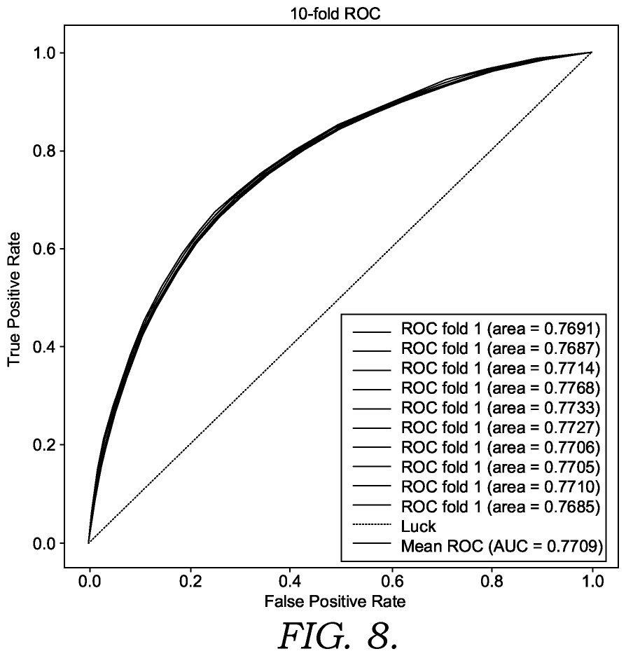

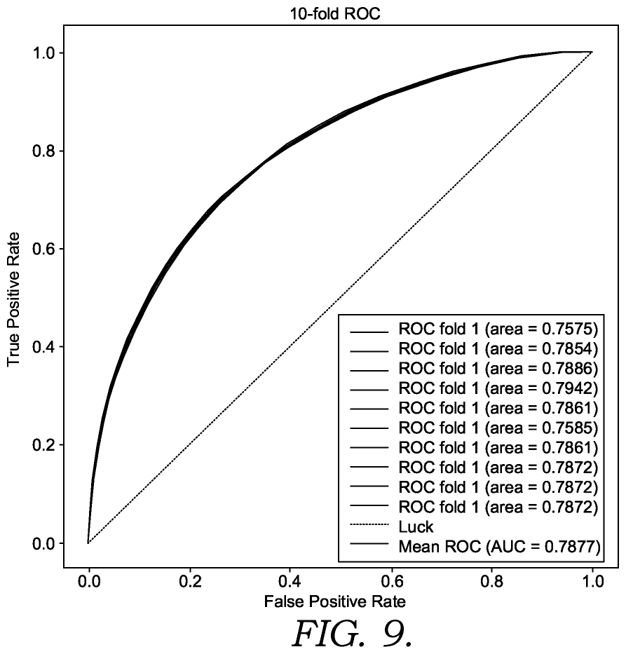

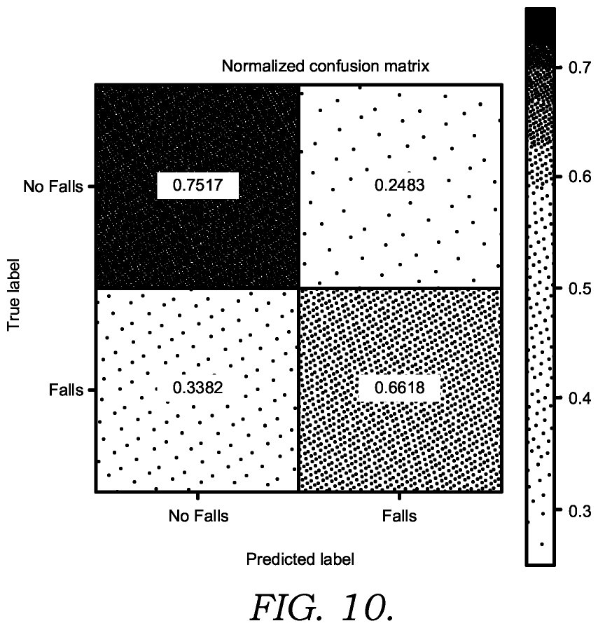

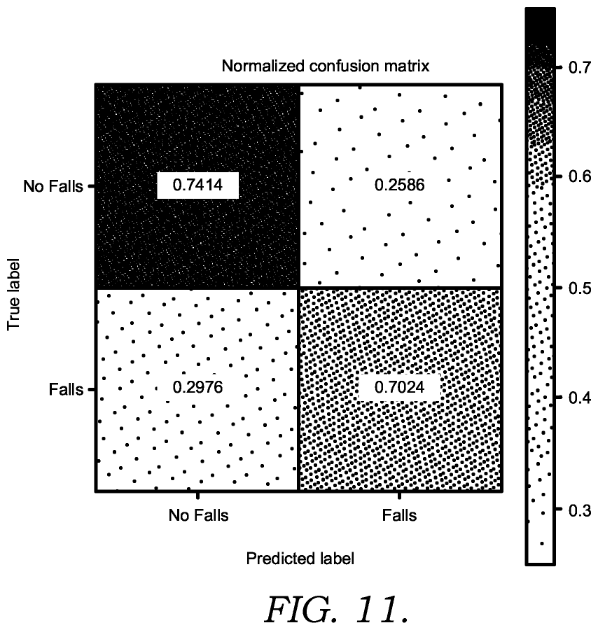

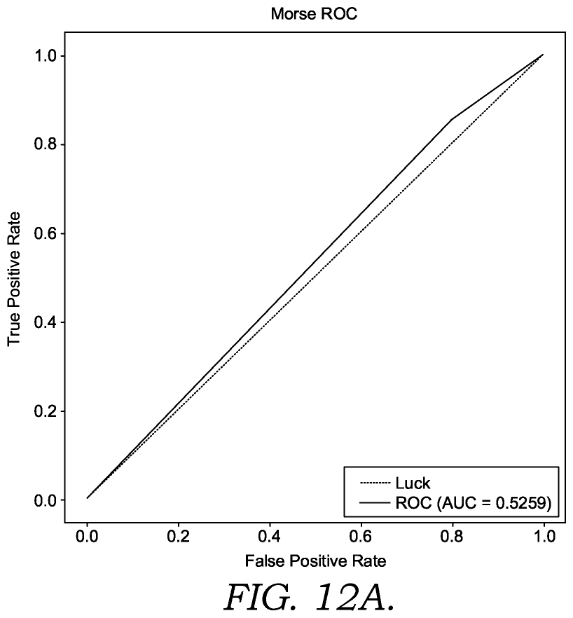

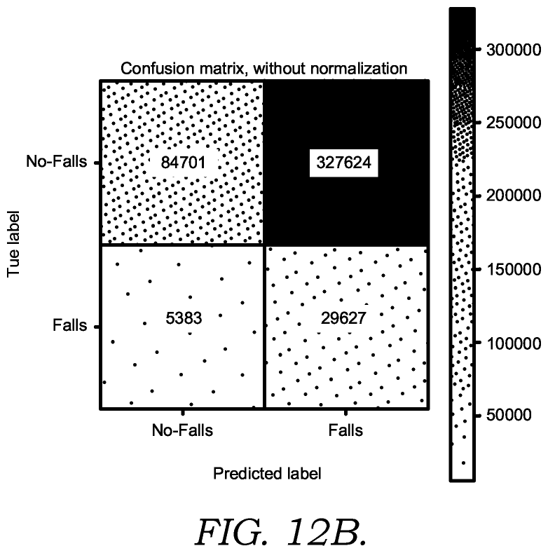

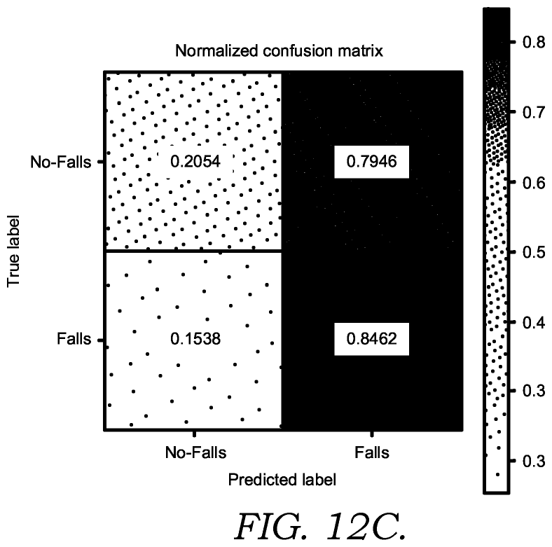

Some embodiments may assess performance beyond a single fold AU-ROC on the test set by performing other techniques, such as a 10-fold cross-validation on a holdout test set of data. In one embodiment actually reduced to practice, the 10-fold cross-validation for a logistic regression model had a mean AUC-ROC of 0.772 (with a range of 0.769-0.777) as depicted in FIG. 8, and the 10-fold cross-validation for the XGBoost model had a mean AUC-ROC of 0.788 (with a range of 0.787-0.797) as depicted in FIG. 9. These results are greatly improved than when using conventional technologies based on the Morse Falls Risk Assessment, which had an AUC-ROC of 0.55 (as shown in FIG. 12A). FIGS. 12B and 12C show confusion matrices for conventional technologies based on Morse Falls Risk Assessment. Further, in some embodiments actually reduced to practice for patient data limited to a single post-acute care facility, the AUC-ROC was 0.928 for the logistic regression model and 0.920 for the XGBoost model. Additionally, performance is improved over Confusion matrices (based on optimal tpr-fpr) for the logistic regression model and the XGBoost model are depicted in FIG. 10 and FIG. 11, respectively. More detailed performance metrics (Precision, Recall, Fl score) for each model can be found in Table 5 and Table 6, below:

TABLE 5
Logistic Regression Confusion Matrix

| | precision | recall | F1-score | support |
|---|---|---|---|---|
| 0 | 0.9668 | 0.7130 | 0.8207 | 198318 |
| 1 | 0.1676 | 0.7025 | 0.2706 | 16313 |
| accuracy | | | 0.7122 | 214631 |
| macro avg | 0.5672 | 0.7078 | 0.5457 | 214631 |
| weighted avg | 0.9061 | 0.7122 | 0.7789 | 214631 |

TABLE 6
XGBoost Confusion Matrix

| | precision | recall | F1-score | support |
|---|---|---|---|---|
| 0 | 0.9688 | 0.7374 | 0.8374 | 198318 |
| 1 | 0.1822 | 0.7116 | 0.2902 | 16313 |
| accuracy | | | 0.7354 | 214631 |
| macro avg | 0.5755 | 0.7245 | 0.5638 | 214631 |
| weighted avg | 0.9090 | 0.7354 | 0.7958 | 214631 |

### Performance on Subsets (Bias Analysis)

To assess any bias of the fall risk model and sure it outputs useful predictions regardless of race or financial class, performance for each cohort may be considered. In one embodiments actually reduced to practice, there was a difference in performance in each racial group, but the performance appears to be within an minimal range (0.013 AU-ROC for both logistic regression and XGBoost) between the group with the highest and lowest performance (see Table 7 and Table 8, below).

TABLE 7
Logistic Regression Model Performance by Race/Ethnicity

| | | Outcome | | |
|---|---|---|---|---|
| | Race % | % | AUC | PRAUC |
| White | 77.7 | 7.48 | 0.768 | 0.245 |
| Black | 13 | 8.55 | 0.777 | 0.288 |
| Hispanic | 4.62 | 7.43 | 0.777 | 0.245 |
| Other | 4.72 | 8.04 | 0.764 | 0.242 |
| Overall | 100 | 7.64 | 0.770 | 0.250 |

TABLE 8
XGBoost Model Performance by Race/Ethnicity

| | | Outcome | | |
|---|---|---|---|---|
| | Race % | % | AUC | PRAUC |
| White | 77.7 | 7.48 | 0.791 | 0.293 |
| Black | 13 | 8.55 | 0.804 | 0.334 |
| Hispanic | 4.62 | 7.43 | 0.802 | 0.283 |
| Other | 4.72 | 8.04 | 0.798 | 0.315 |
| Overall | 100 | 7.64 | 0.794 | 0.299 |

Model performance by financial class had little variance (0.011 AU-ROC for logistic regression, 0.006 AU-ROC for XGBoost) between groups (see Table 9 and Table 10, below). In some embodiments, the model performance may be adjusted to improve against bias.

TABLE 9
Logistic Regression Model Performance by Financial Class

| | Prevalence | Outcome | | |
|---|---|---|---|---|
| | % | % | AUC | PRAUC |
| Other/Unknown | 28.9 | 8.87 | 0.768 | 0.277 |
| Medicare/Medicaid | 64.7 | 6.91 | 0.777 | 0.230 |
| Private Insurance | 6.39 | 9.48 | 0.759 | 0.298 |
| Overall | 100 | 7.64 | 0.770 | 0.250 |

TABLE 10
XGBoost Model Performance by Financial Class

| | Prevalence | Outcome | | |
|---|---|---|---|---|
| | % | % | AUC | PRAUC |
| Other/Unknown | 28.9 | 8.87 | 0.797 | 0.322 |
| Medicare/Medicaid | 64.7 | 6.91 | 0.792 | 0.281 |
| Private Insurance | 6.39 | 9.48 | 0.791 | 0.341 |
| Overall | 100 | 7.64 | 0.794 | 0.299 |

### Feature Importance (for Individual Predictions)

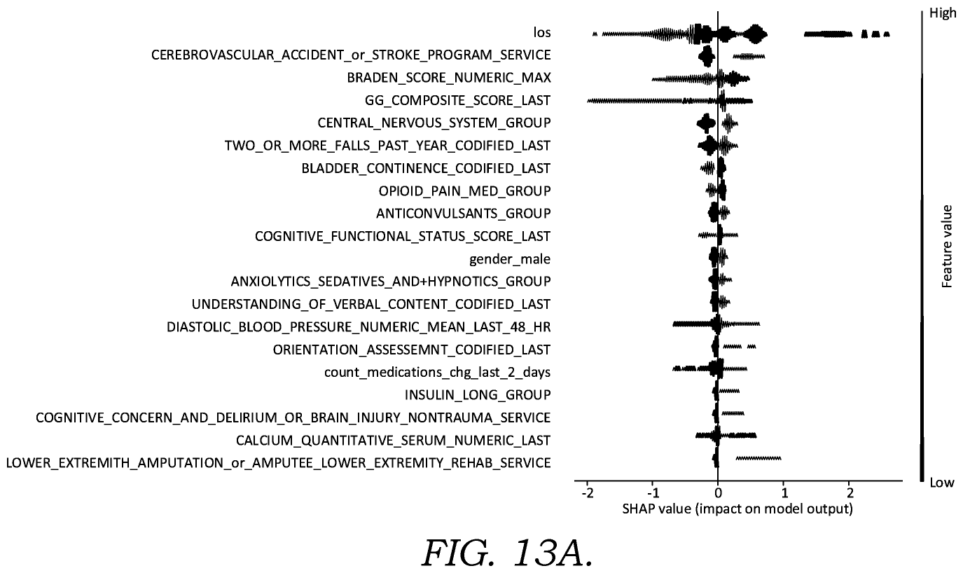

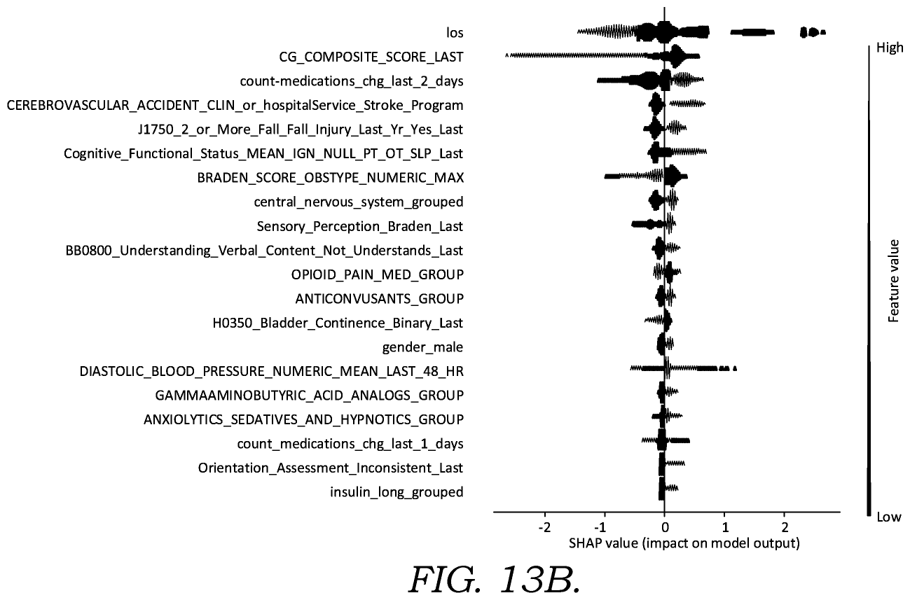

In some embodiments, there are two different fall risk prediction models (Logistic Regression and XGBoost) that can be used interchangeably in production with the same transformed feature set. Table 11 below details some example features ranked in order by the absolute values of their coefficients. Additionally, Table 11 includes the mean values for example Fall and Non-Fall cohorts to help illustrate how the two cohorts vary. The mean values represent prevalence between zero and one for the binary values. FIG. 13A depicts top SHAP values for each feature in terms of total gain, which represents the sum each feature's absolute contribution to the XGBoost model, for feature selection for one embodiment of the fall risk prediction model. FIG. 13B depicts top SHAP values for each feature for another embodiment of the XGBoost fall risk prediction model.

TABLE 11
Logistic Regression Model Features

| | | | Non- | |
|---|---|---|---|---|
| | | | Fall | Fall |
| rank | Falls Risk Model Feature | Coeff | Mean | Mean |
| 1 | los | −0.5781 | 6.4535 | 3.885 |
| 2 | LEVEL_OF_CONSCIOUSNESS_CODIFIED_LAST_or_{—} | −0.3018 | 0.9997 | 0.9989 |
| | HALLUCINATIONS_PRESENT_CODIFIED_LAST | | | |
| 3 | CEREBROVASCULAR_ACCIDENT_or_STROKE_{—} | 0.2721 | 0.21 | 0.3261 |
| | PROGRAM_SERVICE | | | |
| 4 | BRADEN_SCORE_NUMERIC_MAX | −0.2364 | 18.323 | 17.526 |
| 5 | CENTRAL_NERVOUS_SYSTEM_GROUP | 0.1761 | 0.5294 | 0.6253 |
| 6 | BLADDER_CONTINENCE_CODIFIED_LAST | −0.1634 | 0.6259 | 0.4589 |
| 7 | count_medications_chg_last_1_days | 0.1627 | 0.2973 | 4.0397 |
| 8 | GG_COMPOSITE_SCORE_LAST | −0.1432 | 65.9264 | 52.3052 |
| 9 | TWO_OR_MORE_FALLS_PAST_YEAR_CODIFIED_LAST | 0.1153 | 0.5318 | 0.584 |
| 10 | ORIENTATION_ASSESSMENT_CODIFIED_LAST | 0.0993 | 0.0901 | 0.1719 |
| 11 | LOWER_EXTREMITY_AMPUTATION_or_AMPUTEE_{—} | 0.0967 | 0.0339 | 0.0479 |
| | LOWER_EXTREMITY_REHAB_SERVICE | | | |
| 12 | BOWEL_CONTINENCE_STATUS_CODIFIED_LAST | −0.0957 | 0.7233 | 0.5778 |
| 13 | ANTICONVULSANTS_GROUP | 0.0919 | 0.4375 | 0.5009 |
| 14 | OPIOID_PAIN_MED_GROUP | −0.0826 | 0.5356 | 0.4403 |
| 15 | gender_male | 0.0807 | 0.4758 | 0.539 |
| 16 | SPINAL_CORD_NONTRAUMA_SERVICE_or_SPINAL_{—} | 0.0761 | 0.0378 | 0.0437 |
| | CORD_INJURY_TRAUMA_SERVICE | | | |
| 17 | DIASTOLIC_BLOOD_PRESSURE_NUMERIC_MEAN_{—} | 0.0717 | 71.2224 | 72.5124 |
| | LAST_48_HR | | | |
| 18 | count_medications_chg_last_2_days | 0.0706 | 2.9038 | 7.1524 |
| 19 | COGNITIVE_CONCERN_AND_DELIRIUM_or_BRAIN_{—} | 0.0631 | 0.1286 | 0.1468 |
| | INJURY_NONTRAUMA_SERVICE | | | |
| 20 | ADAMANTANE_ANTIVIRALS_GROUP | 0.0615 | 0.0103 | 0.0235 |
| 21 | UNDERSTANDING_OF_VERBAL_CONTENT_{—} | 0.0569 | 0.4385 | 0.5767 |
| | CODIFIED_LAST | | | |
| 22 | LAXATIVES_GROUP | −0.054 | 0.7198 | 0.6561 |
| 23 | ANXIOLYTICS_SEDATIVES_AND_HYPNOTICS_GROUP | 0.0537 | 0.4144 | 0.4312 |
| 24 | INSULIN_LONG_GROUP | 0.0535 | 0.1577 | 0.1908 |
| 25 | COGNITIVE_FUNCTIONAL_STATUS_SCORE_LAST | −0.0528 | 2.6393 | 2.9201 |
| 26 | D03145 | −0.0484 | 0.176 | 0.1528 |
| 27 | D00070 | −0.0471 | 0.2277 | 0.1867 |
| 28 | D03130 | 0.0445 | 0.0482 | 0.0681 |
| 29 | GAMMAAMINOBUTYRIC_ACID_ANALOGS_GROUP | 0.0425 | 0.2967 | 0.3198 |
| 30 | ANTIARRHYTHMIC_GROUP | 0.0425 | 0.0757 | 0.0776 |
| 31 | D03821 | −0.0419 | 0.1554 | 0.1381 |
| 32 | D00278 | −0.0405 | 0.2176 | 0.1957 |
| 33 | CALCIUM_QUANTITATIVE_SERUM_NUMERIC_LAST | 0.0405 | 8.8585 | 8.9051 |
| 34 | ALBUTEROL_IPRATROPIUM | −0.0401 | 0.0933 | 0.0774 |
| 35 | ANTICOAGULANTS_GROUP | −0.0372 | 0.6435 | 0.6087 |
| 36 | TRAUMATIC_BRAIN_INJURY | 0.0356 | 0.038 | 0.046 |
| 37 | D00986 | 0.0328 | 0.0062 | 0.0092 |
| 38 | D04283 | −0.0318 | 0.0754 | 0.0577 |
| 39 | ANTIHISTAMINES_GROUP | −0.0288 | 0.1573 | 0.1309 |
| 40 | D00683 | −0.0284 | 0.1414 | 0.105 |
| 41 | D03129 | −0.027 | 0.2174 | 0.1941 |
| 42 | ALBUMIN_NUMERIC_MAX | 0.025 | 3.209 | 3.2396 |
| 43 | ANALGESICS_GROUP | −0.0236 | 0.8958 | 0.8609 |
| 44 | MISCELLANEOUS_ANALGESICS_GROUP | −0.0224 | 0.5313 | 0.4609 |
| 45 | D00316 | 0.0208 | 0.0426 | 0.0544 |
| 46 | ALT_QUANTITATIVE_SERUM_NUMERIC_LAST_LOG | 0.0205 | 2.9851 | 3.0269 |
| 47 | AFFECT_CODIFIED_LAST | 0.0169 | 0.0302 | 0.0604 |
| 48 | SENSORY_BRADEN_CODIFIED_LAST | 0.0113 | 0.7468 | 0.8263 |
| 49 | DIASTOLIC_BLOOD_PRESSURE_NUMERIC_{—} | 0.011 | 0.0394 | 0.0651 |
| | MEDIAN_CAPTURE | | | |
| 50 | D01027 | −0.0106 | 0.025 | 0.0171 |
| 51 | ANTIINFECTIVE_MEDICATIONS | −0.0055 | 0.3699 | 0.342 |
| 52 | TOTAL_DEPRESSION_SCREEN_SCORE_{—} | 0.0041 | 5.304 | 5.7158 |
| | NUMERIC_LAST | | | |
| 53 | HEMATOCRIT_BLOOD_NUMERIC_MEAN_{—} | 0.0035 | 32.9079 | 34.0293 |
| | LAST_48_HR | | | |
| 54 | ANTIEMETICANTIVERTIGO_AGENTS_GROUP | −0.0021 | 0.2511 | 0.2213 |
| 55 | APPETITE_STATUS_CODIFIED_LAST | −0.002 | 2.7023 | 2.6134 |
| 56 | QUINOLONES_GROUP | 0.001 | 0.0795 | 0.0624 |

### Fall Risk Prediction Model Deployment

Some embodiments of the fall risk prediction model may be deployed using a machine learning ecosystem that includes several implementation designs based on the needs of the model, including batch, asynchronous near real-time, and using ccl programs for data extraction. In some embodiments, the fall risk prediction model is developed to have a minimum latency, which as eight hours or less, which would generally match shift changes and therefore be more useable in a post-acute care facility.

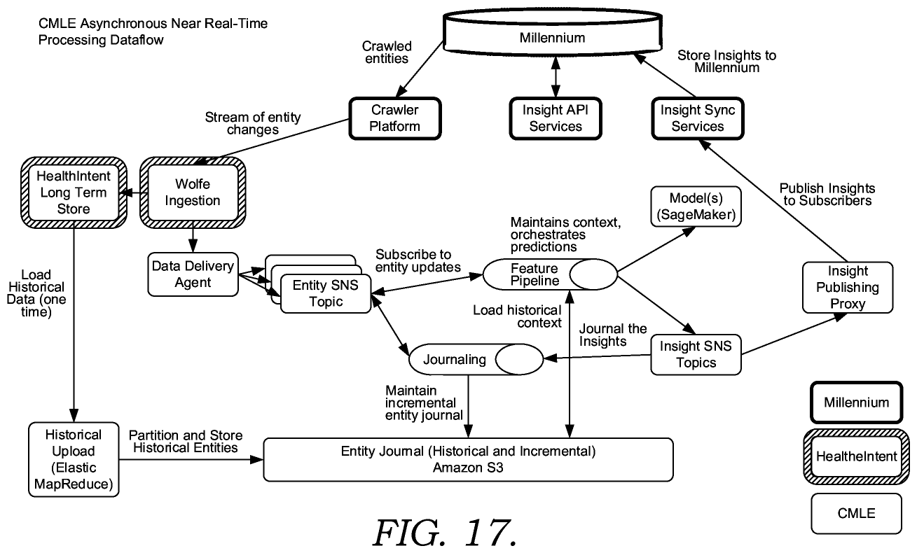

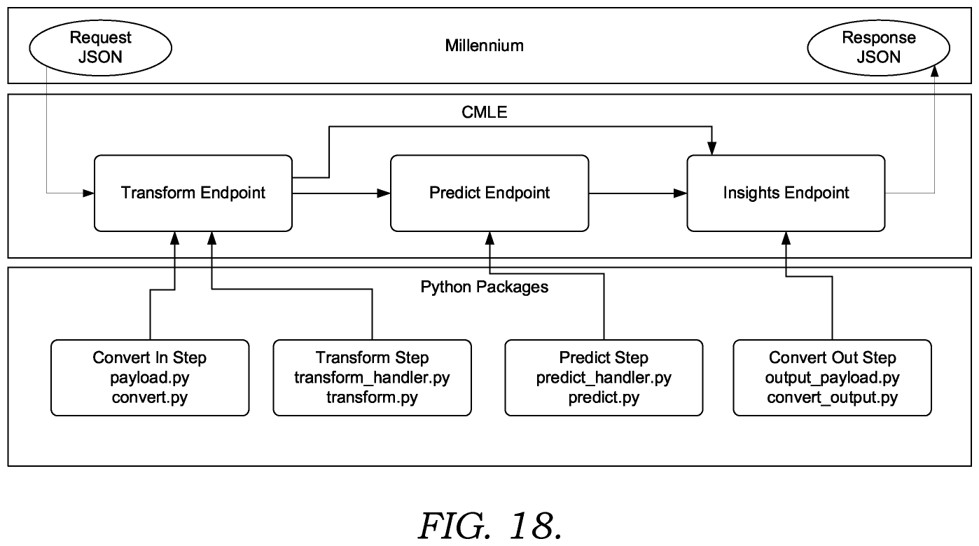

In some embodiments, an asynchronous near real-time processing dataflow is used for the fall risk prediction model. FIG. 17 generally discloses an example asynchronous near real-time processing dataflow pipeline. In accordance with the pipeline in FIG. 17, a near real-time data streaming pipeline brings new patient data into the fall risk prediction model. Discern ontology concepts are leveraged to make the fall risk model more generalizable to other clients. The model may be deployed in a machine learning web service (such as Amazon Web Service (AWS) Sagemaker endpoints), and the prediction results may be written back to Millennium via a publishing proxy and served in real time for any workflow interventions. FIG. 18 generally shows the flow within the model endpoints and python packages.

This pipeline in FIG. 17 starts by using Apache Beam and the Millennium crawler and return raw data with Ontology concept aliases in a nested JSON format. A feature pipeline and model python package created in accordance with embodiments of the disclosure then transforms the nested JSON structure and fit the pre-trained model using three endpoints, which may be endpoints from Sagemaker or another machine learning web service. FIG. 18 shows transform endpoint, prediction endpoint, and insight endpoint. The output of Insights endpoint may be an Insights JSON containing risk scores and interpretation produced by the fall risk prediction model along with covariates and supporting facts. The deployment system, such as CMLE, may also support a two-endpoint design without an Insights endpoint. In the two-endpoint design, the Predict endpoint may return the same Insights JSON if a client request a mine type of “application/vnd.cerner.cmle+json”. Both two-endpoint and three endpoint designs may be used with the python packages depicted, and reusable code may be used by both two-endpoints and three-endpoint designs. To ensure production code quality, high coverage mock unit testing, functional testing, and code formatting linting tools may be used on the model python package. In some embodiments, the fall risk prediction model and endpoints are created and deployed to a web service, such as Amazon Web Service (AWS) using “model-build” command from the relevant package, such as cmle-sagemaker package. FIG. 22 is another representation of the streaming pipeline in FIG. 17. The encounter, person, diagnosis, and clinical event (e.g., medication administrations, assessments, etc.) may be parallel Beam pipelines.

### Example Software Used

In some embodiments, development of the fall risk prediction model may be done with Python 3 (including pandas, numpy, cipy, XGBoost, and glmnet packages), Apache Spark (for data extraction from Healthe DataLab), Discern Ontologies, and ccl (for Millennium data extraction). The production data extraction may use Wolf Ingestion and AWS Athena. Discern Ontologies may be referenced and returned by the AWS Athena query for use in the model transformations and in the model itself. Output transformations (including the prediction) may use Python 3, cmle-sagemaker, pre-trained models from development (XGBoost, Sci-Kit Learn Logistic Regression), and AWS Sagemaker endpoints—orchestrated by AWS Lambda.

### Employment of Fall Risk Prediction Model for Post-Acute Care Patients

The disclosed fall risk prevention model may be used to predict falls by patients in post-acute care settings, such as in-patient rehabilitation facilities. The model may use information that is readily available from a patient's EMR, billing diagnosis, intake information, and other assessments that are performed in post-acute care facilities. In some embodiments, the data is streamed in near real time such that a patient may be assigned a fall risk level shortly after registration, and this fall risk level may be updated as the prediction changes with changes in the patient's data.

In some aspects, the risk score output by the machine learning model, such as a logistic regression model or XGBoost model, is a probability that the patient will suffer a fall. In some embodiments, the predicted output is a risk category based on the predicted probability. Further, a list of covariates (model features with weights) may be surfaced for the end-user when using some models, such as logistic regression. In other embodiments, a fixed list of feature ranks (in total gain) may be output as supporting facts, such as when using an XGBoost model.

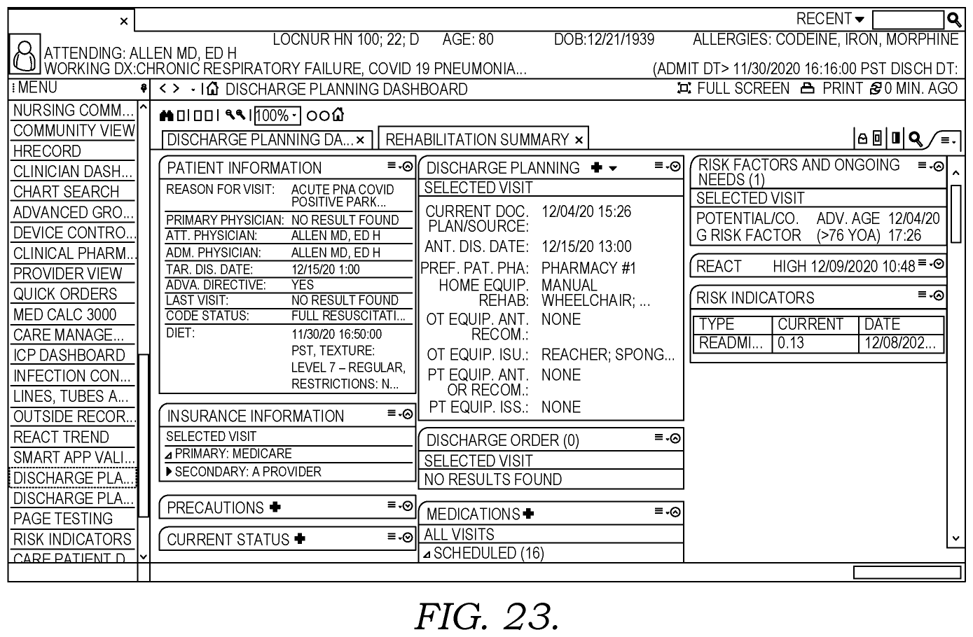

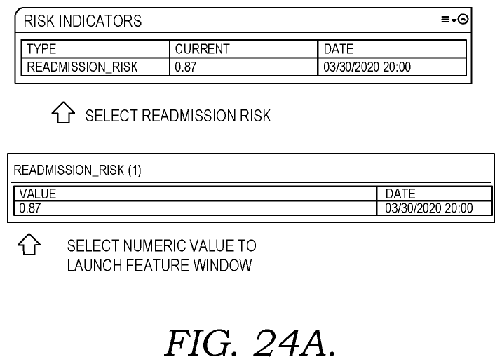

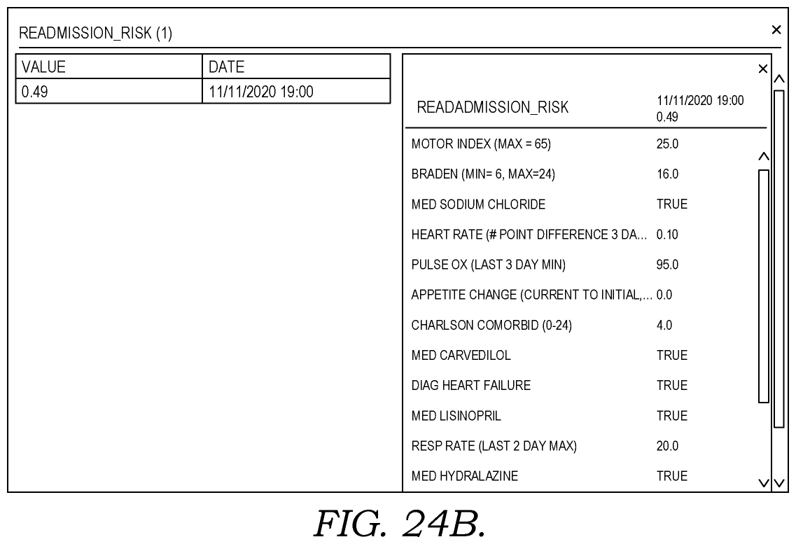

In some embodiments, the fall risk model may output a CSV or JSON file with the predicted risk and contributing feature information (e.g., covariates or feature ranks/gains). In some embodiments, a user interface, which may be a clinician user interface or a patient user interface, may be generated to present the predicted risk and, in some embodiments, contributing feature information. An example user interface is depicted in FIGS. 23 and 24A-B. FIG. 23 shows an example fall risk indicator GUI component generated and provided for display with a patient dashboard, which may be provided to a clinician. FIGS. 24A-B depict example GUI components that may be provided when a user selects (e.g., clicks on or hovers over) a displayed risk score component to receive the contributing features.

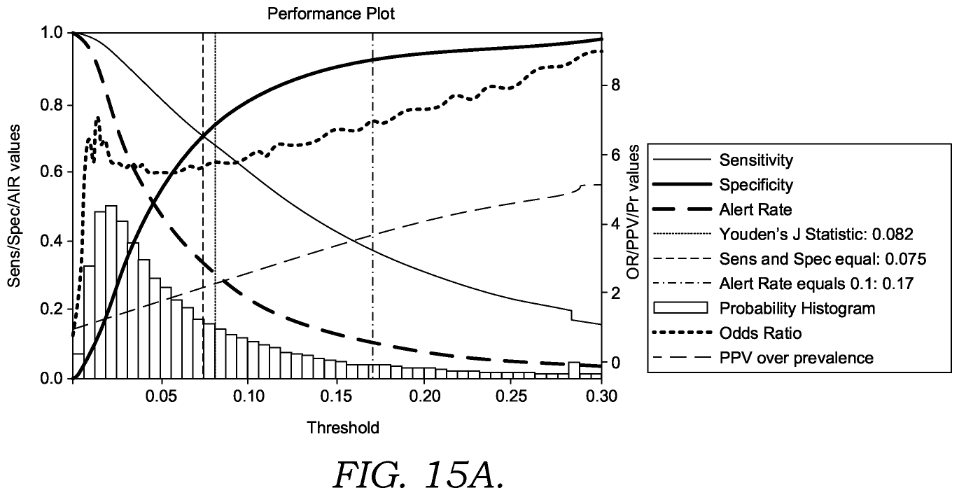

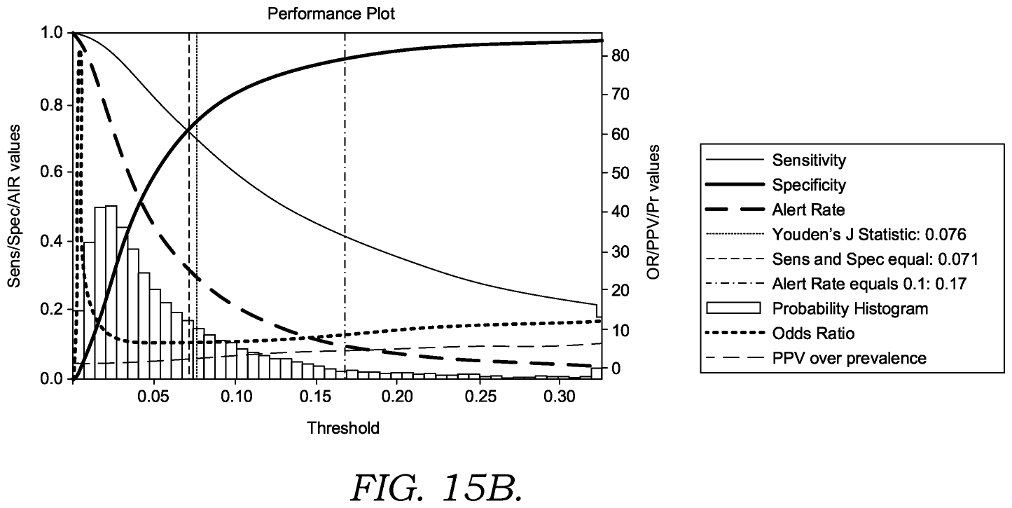

In some aspects, various performance information is output by the system to provide an assessment of the fall risk prediction model. In one example, odds Ratio graphs, optimal F¹ score, Alert Rate, optimal Youden J-Statistic, and Positive Predictive Value (PPV) may be provided and used to determine where to set fall risk threshold(s). FIG. 15A depicts a threshold selection plot for an example logistic regression fall risk prediction model, and FIG. 15B depicts a threshold selection plot for an example XGBoost fall risk prediction model. In one embodiment, the high risk threshold is determined using an optimal Youden Index, while a very high risk threshold is set so that the alert rate is equate to the fall rate in training.

In exemplary aspects, one or more actions may be initiated based on the fall risk prediction. The actions may be actions to mitigate or reduce the risk of the patient falling. In some embodiments, an action to initiate may include emitting or otherwise electronically communicating a recommendation or notification to a caregiver responsible for the patient's care, such as a physician or nurse. This notification may be presented via a user/clinician interface (such as interface 142 described in FIG. 1A and/or as shown in FIG. 23 and FIGS. 24A-B). The notification may indicate the fall prediction of the post-acute care patient, and the notification may present care instructions for the patient, such as increasing monitoring, adding guardrails, increasing particular vitamins, increasing lighting in the patient area, and the like. Additionally, some embodiments of this action may further include storing the result of the fall prediction in an EHR associated with the patient and further may include providing the patient's EHR (or facilitating access to the EHR) in the notification.

An action may include scheduling time(s) for care providers to check in and/or monitor the patient and/or allocating resources, such as rooms on a main level and/or near often used common areas, guard rails, floor mats, lights, and the like that may reduce the risk of a patient having a fall. The action may also include electronically adding one or more documents with the special instructions to a queue associated with the patient's record, which may include a queue designating documents for printing and/or providing to the patient. One or more care providers may be notified of the additional documentation so that the care provider may give it to the patient. Further, additional testing to confirm an increased fall risk may be ordered.

One or more of these actions may be performed by automatically modifying computer code executed in a healthcare software program for treating the patient and/or care planning, thereby transforming the program at runtime. For example in one embodiment, the modification comprises modifying (or generating new) computer instructions to be executed at runtime in the program, the modification may correspond to a change in a care procedure due to the predicted fall risk (e.g., a change in a count of a particular medication administered over a particular lookback period or a change in a dosage of the particular medication). In some aspects, the change in a care procedure may correspond to an action to reduce the patient's risk or likelihood of falling.

In some embodiments, the actions may be initiated automatically when the probability of a fall satisfies a threshold level (i.e., a threshold probability). In some embodiments, the threshold probability may be a value within a range of 0 to 1. In some embodiments, the threshold probability is between 0.175 and 1. For example, in one embodiment, the threshold probability may be 0.2. In some embodiments, the threshold may customizable for a particular clinician or healthcare provider and may be configured by a software application that is separate from but works in conjunction with the software application running the predictive model. Selections for thresholds may include a selection for risk categories, which may each be associated with an action or set of actions. In one embodiment, threshold configurations may include a threshold selection for very high risk, a threshold selection for high risk, a threshold selection for low risk. Additionally, a threshold may be configured based on risk level relative to other patients. For example, a user, such as a clinician or representative of healthcare facility, may select very high risk for a top 7% of patients, high risk for the following 10% of patients, and low risk for the following 83% of patients. In embodiments in which threshold is based on relative risk, thresholds may be configured differently depending on the statistical method used (e.g. a maximum absolute value of a true positive rate minus a false positive rate, or optimal point on an ROC curve). Multiple Falls

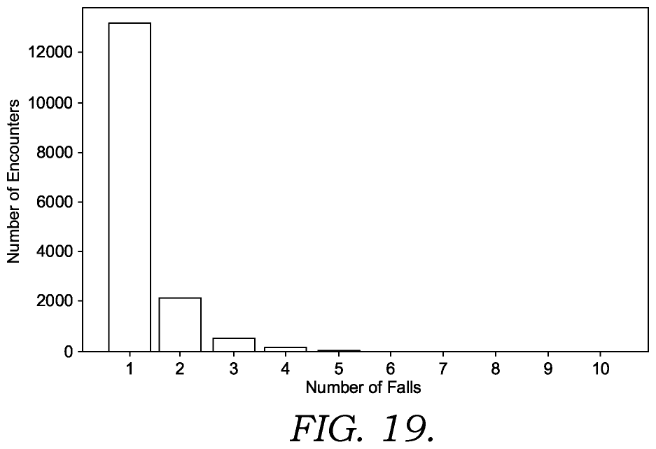

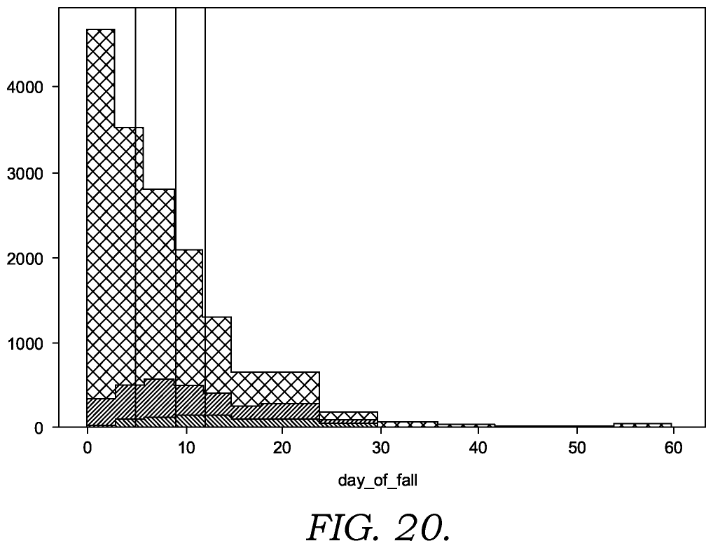

FIG. 19 depicts a graph showing the number of encounters with multiple falls. Out of 16,104 encounters that had a fall event between Jun. 1, 2019 and Jul. 31, 2020 approximately 2,966 encounters had two or more falls. FIG. 20 depicts the distribution of falls based on the day from registration. FIG. 20 shows that, of 20,232 total falls, 51% of first falls (blue) happen by day 5, but only 28% of second falls (brown) happen by day 5. Some embodiments of the disclosure include a fall risk prediction model that predicts a risk of subsequent falls for a post-acute care patient that has already suffered a fall. In some embodiments, this fall risk prediction model is the same model used to predict an initial fall. In other embodiments, a separate model—a subsequent fall risk prediction model—is developed and employed in accordance with the various aspects of this disclosure. For a separate model, the model may be trained in accordance with the previously disclosed training data but patients having only 1 fall may be removed from the population for training purposes.

Many different arrangements of the various components depicted, as well as components not shown, are possible without departing from the spirit and scope of the present invention. It will be understood that certain features and subcombinations are of utility and may be employed without reference to other features and subcombinations and are contemplated within the scope of the claims. Not all steps listed in the various figures need be carried out in the specific order described. Accordingly, the scope of the invention is intended to be limited only by the following claims.

Some example aspects of the technology that may be practiced from the forgoing disclosure include the following:

Aspect 1: A non-transitory computer storage media having computer-executable instructions embodied thereon that, when executed, provide a method for predicting fall risk for a post-acute care patient, the method comprising: receiving patient data for the post-acute care patient; extracting features from the patient data, the features including one or more polypharmacy features; based on the features extracted from the patient data, generating a prediction of the post-acute care patient suffering a fall within the future using one or more machine learning models; and initiating an action based on the prediction of the post-acute care patient suffering the fall.

Aspect 2: Aspect 1, wherein the prediction of the post-acute care patient suffering the fall is generated within 36 hours of the post-acute care patient being admitted into a post-acute care facility.

Aspect 3: Any of Aspects 1-2, wherein the features include a condition, the condition being extracted from a billing diagnoses.

Aspect 4: Any of Aspects 1-3, wherein the features include a condition, the condition being extracted from a free-text form in an electronic health record of the post-acute care patient using one or more natural language processing techniques.

Aspect 5: Any of Aspects 1-4, wherein the patient data includes data from multiple types of the following types: demographics, social determinants of health (SDOH), patient medications, hospital services, billing diagnoses, functional assessments, cognitive assessments, laboratory results, and vitals.

Aspect 6: Any of Aspects 1-5, wherein the patient data includes data from each of the following types: demographics, SDOH, patient medications, hospital services, billing diagnoses, functional assessments, cognitive assessments, laboratory results, and vitals.

Aspect 7: Any of Aspects 1-6, wherein a condition feature is determined by combining hospital service data and billing diagnosis data.

Aspect 8: Any of Aspects 1-7, wherein the one or more polypharmacy features include a count of unique medications, a change in unique medications of a predetermined lookback period, and drug interactions.

Aspect 9: Aspect 8, wherein the drug interactions are identified using an XGBoost model.

Aspect 10: Any of Aspects 1-9, wherein the one or more machine learning models used for generating the prediction of the post-acute care patient suffering the fall is an XGBoost model.

Aspect 11: Any of Aspects 1-10, wherein the one or more machine learning models used for generating the prediction of the post-acute care patient suffering the fall is a logistic regression model.

Aspect 12: A method for predicting fall risk for a post-acute care patient, the method comprising: storing training data associated with a plurality of patients for training one or more machine learning models that include one or more models for generating a prediction of the post-acute care patient suffering a fall within the future; extracting features values from patient data of the post-acute care patient from an electronic medical record (EMR) of the post-acute care patient; based on the features values extracted from the patient data, generating the prediction of the post-acute care patient suffering the fall within the future using the one or more machine learning models trained using the training data; determining the prediction of the post-acute care patient suffering the fall is above a threshold; and initiating an action based on the prediction being above the threshold.

Aspect 13: Aspect 12, further comprising standardizing the training data using mappings that map client-specific nomenclature and codes to standard nomenclature and codes and grouping the mapped standard nomenclature and codes into clinical ontology concepts.

Aspect 14: Any of Aspects 12-13, wherein each of the plurality of patients experienced one or more falls during a patient encounter, the method further comprising labeling the training data based on a severity of an injury corresponding to the one or more falls.

Aspect 15: Any of Aspects 12-14, wherein the features values extracted from the patient data correspond to one or more counts and changes in counts of a particular medication administered to the post-acute care patient over one or more lookback periods, drug interactions for the particular medication, and medication features based on a dosage of the particular medication.

Aspect 16: Any of Aspects 12-15, further comprising: separating continuous features values and binary features values of the features values; separating the features values based on a demographic feature, a condition feature, a medication feature, and a result feature; inputting the continuous features values into the trained one or more machine learning models, the continuous features values ordered based on the demographic feature, the condition feature, the medication feature, and the result feature; and inputting the binary features values into the trained one or more machine learning models, the continuous features values ordered based on the demographic feature, the condition feature, the medication feature, and the result feature; wherein the one or more models of the trained one or more machine learning models comprises an XGBoost model.

Aspect 17: A method for generating a fall risk prediction model comprising one or more machine learning models for predicting whether a post-acute care patient will suffer a future fall, the method comprising: receiving patient data for a plurality of patients, wherein at least a subset of the patient data is associated with one or more patients who experienced a fall; identifying features comprising medication features and diagnosis features from the patient data received, wherein at least one of the features are identified using natural language processing from one or more free-text fields within at least one electronic medical record; selecting a subset of the features by: separating continuous values and binary values of the features and using a tree-based model for the continuous values and the binary values separately; identifying permutated features, based on using the tree-based model, that are above a permuted feature total gain threshold; and applying a logistic regression model to the permutated features above the permuted feature total gain threshold; and based on selecting the subset of the features, generating the fall risk prediction model.

Aspect 18: Aspect 17, further comprising identifying a condition feature from the features that comprises a first codified value, for medical service associated with a condition, combined with a second codified value, the second codified value for a diagnosis for the condition.

Aspect 19: Any of Aspects 17-18, further comprising transforming continuous labs features and vitals features of the features from initially entered values into feature values for one or more standardized units of measurement, wherein the continuous values or the binary values comprise the transformed features values.

Aspect 20: Any of Aspects 17-19, wherein at least a portion of the diagnosis features are determined from billing records stored in an electronic medical record.

What is claimed is:

1. A non-transitory computer storage media having computer-executable instructions embodied thereon that, when executed, provide a method for predicting fall risk for a post-acute care patient, the method comprising:
receiving patient data for the post-acute care patient;
extracting features from the patient data, the features including one or more polypharmacy features;
based on the features extracted from the patient data, generating a prediction of the post-acute care patient suffering a fall within the future using one or more machine learning models; and
initiating an action based on the prediction of the post-acute care patient suffering the fall.

2. The non-transitory computer storage media of claim 1, wherein the prediction of the post-acute care patient suffering the fall is generated within 36 hours of the post-acute care patient being admitted into a post-acute care facility.

3. The non-transitory computer storage media of claim 1, wherein the features include a condition, the condition being extracted from a billing diagnoses.

4. The non-transitory computer storage media of claim 1, wherein the features include a condition, the condition being extracted from a free-text form in an electronic health record of the post-acute care patient using one or more natural language processing techniques.

5. The non-transitory computer storage media of claim 1, wherein the patient data includes data from multiple types of the following types: demographics, social determinants of health (SDOH), patient medications, hospital services, billing diagnoses, functional assessments, cognitive assessments, laboratory results, and vitals.

6. The non-transitory computer storage media of claim 1, wherein the patient data includes data from each of the following types: demographics, SDOH, patient medications, hospital services, billing diagnoses, functional assessments, cognitive assessments, laboratory results, and vitals.

7. The non-transitory computer storage media of claim 1, wherein a condition feature is determined by combining hospital service data and billing diagnosis data.

8. The non-transitory computer storage media of claim 1, wherein the one or more polypharmacy features include a count of unique medications, a change in unique medications of a predetermined lookback period, and drug interactions.

9. The non-transitory computer storage media of claim 8, wherein the drug interactions are identified using an XGBoost model.

10. The non-transitory computer storage media of claim 1, wherein the one or more machine learning models used for generating the prediction of the post-acute care patient suffering the fall is an XGBoost model.

11. The non-transitory computer storage media of claim 1, wherein the one or more machine learning models used for generating the prediction of the post-acute care patient suffering the fall is a logistic regression model.

12. A method for predicting fall risk for a post-acute care patient, the method comprising:
storing training data associated with a plurality of patients for training one or more machine learning models that include one or more models for generating a prediction of the post-acute care patient suffering a fall within the future;
extracting features values from patient data of the post-acute care patient from an electronic medical record (EMR) of the post-acute care patient;
based on the features values extracted from the patient data, generating the prediction of the post-acute care patient suffering the fall within the future using the one or more machine learning models trained using the training data;
determining the prediction of the post-acute care patient suffering the fall is above a threshold; and
initiating an action based on the prediction being above the threshold.

13. The method of claim 12, the method further comprising standardizing the training data using mappings that map client-specific nomenclature and codes to standard nomenclature and codes and grouping the mapped standard nomenclature and codes into clinical ontology concepts.

14. The method of claim 13, wherein each of the plurality of patients experienced one or more falls during a patient encounter, the method further comprising labeling the training data based on a severity of an injury corresponding to the one or more falls.

15. The method of claim 12, wherein the features values extracted from the patient data correspond to one or more counts and changes in counts of a particular medication administered to the post-acute care patient over one or more lookback periods, drug interactions for the particular medication, and medication features based on a dosage of the particular medication.

16. The method of claim 15, further comprising:
separating continuous features values and binary features values of the features values;
separating the features values based on a demographic feature, a condition feature, a medication feature, and a result feature;
inputting the continuous features values into the trained one or more machine learning models, the continuous features values ordered based on the demographic feature, the condition feature, the medication feature, and the result feature; and
inputting the binary features values into the trained one or more machine learning models, the continuous features values ordered based on the demographic feature, the condition feature, the medication feature, and the result feature;
wherein the one or more models of the trained one or more machine learning models comprises an XGBoost model.

17. A method for generating a fall risk prediction model comprising one or more machine learning models for predicting whether a post-acute care patient will suffer a future fall, the method comprising:
receiving patient data for a plurality of patients, wherein at least a subset of the patient data is associated with one or more patients who experienced a fall;
identifying features comprising medication features and diagnosis features from the patient data received, wherein at least one of the features are identified using natural language processing from one or more free-text fields within at least one electronic medical record;
selecting a subset of the features by:
separating continuous values and binary values of the features and using a tree-based model for the continuous values and the binary values separately;
identifying permutated features, based on using the tree-based model, that are above a permuted feature total gain threshold; and
applying a logistic regression model to the permutated features above the permuted feature total gain threshold; and
based on selecting the subset of the features, generating the fall risk prediction model.

18. The method of claim 17, further comprising identifying a condition feature from the features that comprises a first codified value, for medical service associated with a condition, combined with a second codified value, the second codified value for a diagnosis for the condition.

19. The method of claim 17, further comprising transforming continuous labs features and vitals features of the features from initially entered values into feature values for one or more standardized units of measurement, wherein the continuous values or the binary values comprise the transformed features values.

20. The method of claim 17, wherein at least a portion of the diagnosis features are determined from billing records stored in an electronic medical record.
